(19) United States
(12) Patent Application Publication
(10) Pub. No.: US 2023/0329982 A1
(43) Pub. Date: Oct. 19, 2023

(54) RESIN COMPOSITIONS COMPRISING A MACROMOLECULE CROSSLINKED WITH POLYAMIDE EPICHLOROHYDRIN, CONTROLLED RELEASE PARTICLES AND COMPOSITIONS COMPRISING SAME, AND METHODS OF MAKING SAME

(71) Applicant: TRUCAPSOL LLC, Bethlehem, PA (US)

(72) Inventors: Praveen BACHAWALA, Allentown, PA (US); Jiten DIHORA, Center Valley, PA (US)

(21) Appl. No.: 17/724,166

(22) Filed: Apr. 19, 2022

(51) Int. Cl. A61K 8/11; A61Q 19/00; A01N 25/28; A01P 7/04; C08G 81/00; B01J 13/10; B01J 13/16; C11D 11/00; C11D 3/00; C11D 3/50

(52) U.S. Cl. CPC A61K 8/11; A61Q 19/00; A01N 25/28; A01P 7/04; C08G 81/00; B01J 13/10; B01J 13/16; C11D 11/0017; C11D 3/0015; C11D 3/505; A61K 2800/10; A61K 2800/654

(57) ABSTRACT

Disclosed is a resin composition including a macromolecule crosslinked with polyamide epichlorohydrin, wherein the macromolecule is a polypeptide, a protein, a polysaccharide, an oligosaccharide, a polyphenol and/or a lipid. Further disclosed is a controlled release composition having a plurality of particles including: a core having at least one hydrophobic active ingredient and the resin composition; and a shell at least partially surrounding the core and effective to inhibit diffusion of the at least one hydrophobic active ingredient into an environment surrounding the controlled release composition. The controlled release composition is preferably a consumer product. A method for preparing the compositions is also disclosed.

### BACKGROUND OF THE INVENTION

### 1. Field of Invention

This invention relates to controlled release compositions, encapsulation compositions and methods for making and using them.

### 2. Description of Related Art

There are many microencapsulated delivery systems disclosed in the art to control the release of the encapsulated active, or provide release when a specific trigger is applied. Such systems have previously suffered from a number of drawbacks.

Controlled release microcapsules that provide release of active upon application of shear or friction generally suffer from several drawbacks: (1) such microcapsules are made of highly crosslinked membranes and membrane materials that cannot be broken down by microbes found in the environment, (2) despite such highly crosslinked membranes, the materials of construction of the membrane impart high permeabilities when incorporated into products that contain high levels of surfactant, solvents, and/or water, which results in the premature benefit agent release, (3) they can only effectively encapsulate a limited breadth of benefit agents, (4) they either are so stable that they do not release the benefit agent in use or have insufficient mechanical stability to withstand the processes required to incorporate them in and/or make a consumer product, (5) they do not adequately deposit on the surface that is being treated with consumer product that contains microcapsules, and/or (6) they do not comprise membrane materials that have a favorable environmental degradability profile.

Such microcapsules are made via chemical processes that require the development of a membrane at the oil-water interface. Said membrane can be developed from the oil side or the water side, or both. An emulsion comprising the active material (dispersed phase) is stabilized in a continuous phase. In one mode, a shell material is deposited from the continuous phase onto a dispersed phase via precipitation of the shell material. In another mode, the shell material is manufactured within the dispersed phase, and migration of the shell material is induced via an interfacial reaction or insolubility of the shell material in the oil phase. The two approaches could be combined to develop “multi-shell” capsules.

In general, conventional microcapsules have a structure of an encapsulated active composition enclosed in a polymeric shell. The microcapsules have a polymeric shell which is generally obtained by any one of: (a) condensation reactions, (b) free radical polymerization reactions, (c) interfacial polymerization reactions, or (d) coacervation of pre-formed polymers followed by crosslinking of the thereby obtained coacervates by using a crosslinker.

There is a challenge in designing a polymeric shell membrane that minimizes the diffusion of the encapsulated active into the surrounding formulation, and yet is environmentally biodegradable. Environmentally biodegradable polymers generally swell in water, or are soluble in water. In contrast, microcapsule membranes generally need to resist swelling or dissolution in aqueous cleaning product formulation. A high degree of crosslinking within the membrane can reduce swelling and solubility; however, such highly crosslinked membranes are difficult for environmentally available microbes to digest and breakdown.

There are four main types of core/shell microcapsules commercialized in industry: aminoplast made via condensation, polyurea made via interfacial polymerization, polyacrylate made via free radical polymerization, and complex coacervate capsules made via hardening a coacervate of gelatin and gum Arabic. Aminoplast capsules comprise a core of hydrophobic active material surrounded by a polyurea shell. The shell is the result of a condensation reaction of methylolated urea or methylolated melamine catalyzed by acidic conditions. U.S. Pat. No. 8,357,651B2 (Givaudan), U.S. Pat. No. 7,122,503B2 (Appleton Papers), GB1502440A (Moore Business Forms), and U.S. Pat. No. 9,359,464B2 (Firmenich) provide detailed information on the preparation of such capsules.

Polyurea capsules made via interfacial polymerization of isocyanates and amines are disclosed in WO2020195132A1. The application discloses polyisocyanates dissolved in an oil phase, and the amines dissolved in the water phase. These two materials come together at the oil/water interface to produce a polyurea reaction product. The capsules have porous shells that cause premature leakage of the encapsulated active, and such polyurea membranes have less than 30% environmental biodegradability (OECD 301D, 60 days). US20130089590A1 and US20120148644A1 also provide details on the preparation of polyurea capsules via the use of isocyanates.

Polyacrylate capsules made via free radical polymerization are disclosed in U.S. Pat. No. 9,937,477B2. The patent discloses core/shell microcapsules that are manufactured using free radical polymerization of acrylates. Such microcapsules require multi-step reactions that require heating the capsules to 95° C. for up to 6 hours. It is well known that such highly crosslinked polyacrylate shells have poor environmental biodegradability. U.S. Pat. No. 8,071,214B2 (Encapsys) also provides details on preparation of acrylate capsules via free radical polymerization.

Complex coacervate capsules are disclosed in U.S. Pat. No. 6,544,926B1 (Encapsys).

While others have attempted to improve the barrier properties of microcapsules, there remains significant shortcomings and limitations in the art. For example, U.S. Pat. No. 9,944,886B2 to Hitchcock et. al. describes metal coated microcapsules with improved barrier properties. The Hitchcock metal coating is developed after the formation of the microcapsule membrane, via the use of a sterically stabilized nanosuspension of metal particle. Such metal coated microcapsules could improve barrier properties; however, it is difficult to imagine how the encapsulated active would be released, since a metal coating would be difficult to fracture. Furthermore, the processing steps involved to achieve the metal coating are laborious and expensive. Moreover, such metal coating could render the microcapsules non-environmentally biodegradable.

Conventional controlled release particles that comprise a core and a shell have several limitations. First, such capsules prematurely release the active material when suspended in a finished product formulations, such as cleaning product formulations. Second, such capsules have poor environmental biodegradability due to the nature of materials used and the degree of crosslinking that is achieved in order to reduce the diffusion of the active. Third, it is difficult to control the release profile of the encapsulated active. Fourth, poor adhesion of particles to the substrate result in significant loss of the particles, especially when formulations containing such particles are used in rinse-off applications. Examples of such applications include laundering fabrics, shampooing hair, conditioning hair, cleansing the skin, showering, and the like. In such applications, a composition comprising microcapsules is applied to a substrate to initiate cleaning, and subsequently the composition is removed by using water.

Accordingly, it is desired to remove soil and dirt, but desired to retain active materials during the rinsing process by the retention of microcapsules on the substrate.

It is further desired to provide a means to manipulate the release profile of the encapsulated active.

It is further desired to provide microcapsules whose membrane has an environmental biodegradability greater than 50%, achievable with very minor changes in existing commercial scale processes to make such microcapsules.

Hence, it is desired to provide low permeability microcapsules that are able to retain the encapsulated active in surfactant containing solutions, or under highly dilute aqueous conditions. It is further desired to improve the adhesion of microcapsules onto the desired substrate during rinse-off applications. It further is desired to release the encapsulated active in larger quantities, and over a longer duration of time. It is further desired to have capsules that have a favorable environmental biodegradability profile as defined by OECD 301D method (OECD 1992, Test No. 301 Ready Biodegradability, OECD Guidelines for the Testing of Chemicals, Section 3, OECD Publishing, Paris, https://doi.org/10.1787/9789264070349-en).

All references cited herein are incorporated herein by reference in their entireties. The citation of any reference is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

### BRIEF SUMMARY OF THE INVENTION

The present invention relates to microcapsules comprising a core and shell, wherein the shell comprises a membrane developed around the core material to reduce the diffusion of core material into the environment. Materials and methods are presented to seal the pores in the membrane while also improving environmental biodegradability.

The inventors have surprisingly found that incorporation of biodegradable resins in the core material during capsule making achieves a membrane with better barrier properties and better environmental biodegradability. The inventors have discovered that such biodegradable resins need to be modified prior to incorporation into the core, such modifications make them reactive. In the absence of such modification, the biodegradable resins are simply dispersed in the core material, but do not become a part of the membrane surrounding the core material. It is only when these modified resins become a part of the membrane that they impart better barrier properties and better environmental biodegradability.

Accordingly, a first aspect of the invention is a resin composition comprising a macromolecule crosslinked with polyamide epichlorohydrin, wherein the macromolecule is at least one member selected from the group consisting of a polypeptide, a protein, a polysaccharide, an oligosaccharide, a polyphenol and a lipid.

In certain embodiments, the resin composition further comprises an inorganic solid.

In certain embodiments, the resin composition further comprises an inorganic solid selected from the group consisting of clays, organically modified clays, minerals and water insoluble salts.

In certain embodiments, the resin composition further comprises an inorganic solid, and a weight ratio of the inorganic solid to polyamide epichlorohydrin is from 1:99 to 30:70, such as, e.g., 1:99 or 5:95 or 10:90 or 20:80 or 30:70.

In certain embodiments, a weight ratio of polyamide epichlorohydrin to the macromolecule is from 1:99 to 20:80, such as, e.g., 1:99 or 5:95 or 10:90 or 20:80.

In certain embodiments, the macromolecule is polyphenol.

A second aspect of the invention is a controlled release composition, comprising a plurality of particles comprising: a core comprising at least one hydrophobic active ingredient and a macromolecule selected from the group consisting of a polypeptide, a protein, a polysaccharide, an oligosaccharide, a cellulosic material, a polyphenol and a lipid, wherein the macromolecule is crosslinked with a water-based crosslinking agent that is reactive with at least one of an amine functionality, a carboxyl functionality, hydroxyl functionality, and a thiol functionality; and a shell at least partially surrounding the core and effective to inhibit diffusion of the at least one hydrophobic active ingredient into an environment surrounding the controlled release composition.

In certain embodiments, the controlled release composition is a consumer product selected from the group consisting of a powdered food product, a fluid food product, a powdered nutritional supplement, a fluid nutritional supplement, a fluid fabric enhancer, a solid fabric enhancer, a fluid shampoo, a solid shampoo, a hair conditioner, a body wash, a solid antiperspirant, a fluid antiperspirant, a solid deodorant, a fluid deodorant, a fluid detergent, a solid detergent, a fluid hard surface cleaner, a solid hard surface cleaner, a fluid fabric refresher spray, a diaper, an air freshening product, a nutraceutical supplement, a controlled release fertilizer, a controlled release insecticide, a controlled release dye and a unit dose detergent further comprising a detergent and a water soluble outer film.

In certain embodiments of the controlled release composition, the particles have a diameter from 0.1 microns to less than 200 microns.

In certain embodiments, the controlled release composition further comprises at least one suspension agent effective to suspend the particles, wherein the at least one suspension agent is at least one member selected from the group consisting of a rheology modifier, a structurant and a thickener.

In certain embodiments, the at least one suspension agent has a high shear viscosity, at 20 sec⁻¹ shear rate and at 21° C., of from 1 to 7000 cps and a low shear viscosity, at 0.5 sec⁻¹ shear rate at 21° C., of greater than 1000 cps.

In certain embodiments, the at least one suspension agent is a member selected from the group consisting of polyacrylates, polymethacrylates, polycarboxylates, pectin, alginate, gum arabic, carrageenan, gellan gum, xanthan gum, guar gum, gellan gum, hydroxyl-containing fatty acids, hydroxyl-containing fatty esters, hydroxyl-containing fatty waxes, castor oil, castor oil derivatives, hydrogenated castor oil derivatives, hydrogenated castor wax, perfume oil, and mixtures thereof.

In certain embodiments, the controlled release composition is a fluid having a high shear viscosity, at 20 sec⁻¹ and at 21° C., of from 50 to 3000 cps and a low shear viscosity, at 0.5 sec⁻¹ shear rate at 21° C., of greater than 1000 cps.

In certain embodiments, the controlled release composition comprises at least two different types of friction-triggered controlled release particles effective to release the at least one hydrophobic active ingredient at different rates due to a difference in shell material friability or core material viscosity.

In certain embodiments of the controlled release composition, the at least one hydrophobic active ingredient comprises a mixture of a hydrophobic active and a material selected from the group consisting of brominated oils, epoxidized oils, highly nonpolar oils, hydrophobically modified inorganic particles, nonionic emulsifiers and oil thickening agents.

In certain embodiments of the controlled release composition, the shell is comprised of a material having an Environmental Biodegradability greater than 50%.

In certain embodiments of the controlled release composition, the shell is degradable by microbes found in wastewater streams to release the at least one hydrophobic active ingredient.

In certain embodiments of the controlled release composition, the at least one hydrophobic active ingredient is at least one member selected from the group consisting of a flavorant, a fragrance, a chromogen, a dye, an essential oil, a sweetener, an oil, a pigment, an active pharmaceutical ingredient, a moldicide, a herbicide, a fertilizer, a phase change material, an adhesive, a vitamin oil, a vegetable oil, a triglyceride and a hydrocarbon.

A third aspect of the invention is a method for preparing a composition, said method comprising the following steps: mixing a macromolecule with a polyamide epichlorohydrin to provide a homogeneous suspension in water; adjusting a pH of the homogenous suspension; dehydrating the homogeneous suspension to provide a powder; and heating the powder at a temperature greater than 100° C. for more than 30 minutes to provide the composition, which is the resin composition or controlled release composition of the invention.

In certain embodiments of the method, the composition is the controlled release composition and the method further comprises forming the shell by any one of: (a) condensation reactions, (b) free radical polymerization reactions, (c) interfacial polymerization reactions, or (d) coacervation of pre-formed polymers followed by crosslinking of the thereby obtained coacervates by using a crosslinker.

### DETAILED DESCRIPTION OF PREFERRED EMBODIMENTS OF THE INVENTION

### Glossary

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from the group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, unless otherwise noted, the terms “capsule”, “microcapsule” and “particle” are synonyms, which refer to containers for selectively retaining an active ingredient.

As used herein, unless otherwise noted, the terms “shell,” “membrane” and “wall” are synonyms, which refer to barriers at least partially surrounding the core of the particles of the invention.

As used herein, microcapsules “formed under acidic conditions” means that part of the process of forming the microcapsule involves a step where the pH of the suspension in which the microcapsules form is adjusted into the acidic region (less than 7).

As used herein, microcapsules “formed under basic conditions” means that part of the process of forming the microcapsule involves a step where the pH of the suspension in which the microcapsules form is adjusted into the alkaline region (greater than 7).

As used herein, “an unreacted amount” refers to the amount of a reactant not used up in one or more reaction. “An unreacted amount” can be zero to any amount depending on the amount of reactants added.

As used herein, unless otherwise noted, “alkyl” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups, the alkyl groups may be the same or different.

The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like.

The dimensions and values disclosed herein are not to be understood as being strictly limited to the exact numerical values recited. Instead, unless otherwise specified, each such dimension is intended to mean both the recited value and functionally equivalent range surrounding that value. For example, a dimension disclosed as “40 mm” is intended to mean “about 40 mm”.

As used herein “cleaning and/or treatment compositions” means products comprising fluid laundry detergents, fabric enhancers, laundry and/or rinse additives, fluid dishwashing detergents, fluid hard surface cleaning and/or treatment compositions, fluid toilet bowl cleaners that may or may not be contained in a unit dose delivery product all for consumer, agricultural, industrial or institutional use.

The term “absorbent article” is used herein in a very broad sense including any article able to receive and/or absorb and/or contain and/or retain fluids and/or exudates, especially bodily fluids/bodily exudates. Exemplary absorbent articles in the context of the present invention are disposable absorbent articles.

The term “disposable” is used herein to describe articles, which are not intended to be laundered or otherwise restored or reused as an article (i.e. they are intended to be discarded after a single use and preferably to be recycled, composted or otherwise disposed of in an environmentally compatible manner). Typical disposable absorbent articles according to the present invention are diapers, surgical and wound dressings, breast and perspiration pads, incontinence pads and pants, bed pads as well as absorbent articles for feminine hygiene like sanitary napkins, panty liners, tampons, interlabial devices or the like. Absorbent articles suitable for use in the present invention include any type of structures, from a single absorbent layer to more complex multi-layer structures. Certain absorbent articles include a fluid pervious topsheet, a backsheet, which may be fluid impervious and/or may be water vapor and/or gas pervious, and an absorbent element comprised there between, often also referred to as “absorbent core” or simply “core”.

The term “Sanitary tissue product” or “tissue product” as used herein means a wiping implement for post-urinary and/or post-bowel movement cleaning (toilet tissue products), for otorhinolaryngological discharges (facial tissue products) and/or multi-functional absorbent and cleaning uses (absorbent towels such as paper towel products and/or wipe products). The sanitary tissue products of the present invention may comprise one or more fibrous structures and/or finished fibrous structures, traditionally, but not necessarily, comprising cellulose fibers.

The term “tissue-towel paper product” refers to products comprising paper tissue or paper towel technology in general, including, but not limited to, conventional felt-pressed or conventional wet-pressed tissue paper, pattern densified tissue paper, starch substrates, and high bulk, uncompacted tissue paper. Non-limiting examples of tissue-towel paper products include towels, facial tissue, bath tissue, table napkins, and the like.

“Personal care composition” refers to compositions intended for topical application to skin or hair and can be, for example, in the form of a liquid, semi-liquid cream, lotion, gel, or solid. Examples of personal care compositions can include, but are not limited to, bar soaps, shampoos, conditioning shampoos, body washes, moisturizing body washes, shower gels, skin cleansers, cleansing milks, in-shower body moisturizers, pet shampoos, shaving preparations, etc.

“Bar soap” refers to compositions intended for topical application to a surface such as skin or hair to remove, for example, dirt, oil, and the like. The bar soaps can be rinse-off formulations, in which the product is applied topically to the skin or hair and then subsequently rinsed within minutes from the skin or hair with water. The product could also be wiped off using a substrate. Bar soaps can be in the form of a solid (e.g., non-flowing) bar soap intended for topical application to skin. The bar soap can also be in the form of a soft solid which is compliant to the body. The bar soap additionally can be wrapped in a substrate which remains on the bar during use.

“Rinse-off” means the intended product usage includes application to skin and/or hair followed by rinsing and/or wiping the product from the skin and/or hair within a few seconds to minutes of the application step.

“Ambient” refers to surrounding conditions at about one atmosphere of pressure, 50% relative humidity and about 25° C.

“Anhydrous” refers to compositions and/or components which are substantially free of added or free water.

“Antiperspirant composition” refers to antiperspirant compositions, deodorant compositions, and the like. For example, antiperspirant creams, gels, soft solid sticks, body sprays, and aerosols.

“Soft solid” refers to a composition with a static yield stress of about 200 Pa to about 1,300 Pa. The term “solid” includes granular, powder, bar and tablet product forms.

The term “fluid” includes liquid, gel, paste and gas product forms.

The term “situs” includes paper products, fabrics, garments, hard surfaces, hair and skin.

The term “substantially free of” refers to 2% or less of a stated ingredient. “Free of” refers to no detectable amount of the stated ingredient or thing.

As used herein, the terms “a” and “an” mean “at least one”.

As used herein, the terms “include”, “includes” and “including” are meant to be non-limiting.

Unless otherwise noted, in discussing the commercial applications below, all component or composition levels are in reference to the active portion of that component or composition, and are exclusive of impurities, for example, residual solvents or byproducts, which may be present in commercially available sources of such components or compositions.

Similarly, all percentages and ratios are calculated by weight unless otherwise indicated and are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Advantages of the Invention

Surprisingly, incorporating uniquely modified amine moiety containing polymer in the hydrophobic oil phase during microcapsule making results in a membrane that provides better barrier properties to the membrane to reduce the premature leakage of encapsulated hydrophobic active materials, whilst also providing higher environmental biodegradability. More importantly, the polymer can be incorporated into conventional microcapsule making processes (e.g. aminoplast, interfacial polymerization, free radical polymerization, complex coacervation).

One or more of the following benefits are provided by preferred embodiments of the invention.

The inventive particles' shell material has an environmental biodegradability greater than 50% as measured by the OECD 301D method that utilizes biological oxygen demand as the criteria for measuring degradability. Conventional capsules utilize polymers that may be biodegradable prior to shell formation, but due to the nature of crosslinkers that are used and the chemical structure of the final crosslinked polymer, microbes are no longer able to attach to the polymer or the backbone to sufficiently degrade the shell material. The inventive particles utilize monomers and polymers that retain degradable functional groups even after the crosslinking is complete, such that microbes in the environment are able to digest the shell material.

In order to deliver a consumer noticeable benefit, yet deliver that benefit at a low cost, encapsulation is used to isolate a uniquely different fragrance or flavor active from the non-encapsulated fragrance or flavor that is incorporated into the formulation. Acclamation to a flavor or fragrance requires a much higher concentration of the same fragrance or flavor to achieve noticeability. The invention allows one to encapsulate a uniquely different fragrance or flavor to incorporate into the composition, and achieve noticeability at significantly lower concentrations of the encapsulated active. Improvement of retention of capsules onto the fabric during rinse-off processes also has the potential to reduce cost.

Particles

The invention addresses one or more of the prior art deficiencies described above by providing controlled release particles. The particles are particularly well-suited for use in encapsulation of hydrophobic, nonpolar materials.

The particles are preferably used in a consumer product composition, such as, e.g., a cleaning composition, a fabric care composition and/or a personal care composition.

The particles generally comprise a polyurea shell (“trunk”) linked to macromolecules (“branches”) enabled by the use of the novel resin composition of the invention (sometimes referred to hereinafter as “pre-reacted resin”). The pre-reacted resin preferably has several features that make it uniquely different from a natural material: 1) the pre-reacted resin is water insoluble and stays in the oil phase during capsule making whereas a natural material would generally partition to the water phase during capsule making, 2) the pre-reacted resin has reactive secondary amine functional groups sourced from the polyamide epichlorohydrin as well as acid, thiol, amine, and hydroxyl functional groups from the natural material, 3) the pre-reacted resin maintains greater than 60% biodegradability (OECD 301D) despite having a low quantity of synthetic material and crosslinking in the polymer. Natural materials that generally comprise amine, hydroxyl, carboxyl, and/or thiol functionalities are monosaccharides, oligosaccharides, polysaccharides, amino acids, proteins, celluloses, carboxy modified saccharides and celluloses, and the like.

In certain embodiments, the pre-reacted resin comprises a monomer or polymer having at least one functional group that is capable of reacting with isocyanate, acrylate (via Michael addition reaction), or aldehyde groups. Such pre-reacted resin comprises at least one macromolecule and a crosslinker. The macromolecule is selected from the group consisting of polypeptides, proteins, polysaccharides, oligosaccharides, cellulose, polyphenol, lipids and mixtures thereof. The crosslinker is selected from the group consisting of water-based crosslinking resins that are reactive with amine, carboxyl, hydroxyl, and thiol functionality. Preferably, the crosslinker is polyamide epichlorohydrin that has a high content of secondary amines. In a preferred embodiment, the pre-reacted resin is made by pursuing the following procedure: 1) the macromolecule and optional inorganic solid are mixed with the polyamide epichlorohydrin to make a homogeneous solution in water; 2) the pH of the homogeneous solution is adjusted to optimize conditions for the reaction; 3) the mixture is dehydrated, preferably using a spray drying process, and 4) the resulting powder is heated at a temperature greater than 100° C. for more than 30 minutes to assure crosslinking. Nonlimiting examples of pre-reacted resin comprise polyamide epichlorohydrin reaction products with proteins such as casein, whey protein, soy protein, silk protein, zein protein, and the like; reaction products with oligosaccharides and polysaccharides such as chitosan oligosaccharide, carboxymethyl starch, alginic acid, hyaluronic acid, pectin, glucuronic acid, gum Arabic, and the like; reaction products with celluloses such as carboxymethyl cellulose, microcrystalline cellulose, hydroxyethyl cellulose, hydroxypropyl methyl cellulose, hydroxypropylmethyl cellulose phthalate, hydroxypropyl methyl cellulose acetate succinate, and the like; reaction products with polyphenols such as lignin, tannic acid, and the like; and mixtures thereof.

In certain embodiments, the pre-reacted resin is a composite made by combining polyamide epichlorohydrin, protein, a polyphenol containing material, optionally an inorganic solid in a low water content dough that is extruded into a filament, and ground into a fine powder. Said composite is cured at a temperature above 100° C. for at least 30 minutes to yield the pre-reacted resin.

In certain embodiments, the pre-reacted resin comprises a polymer having a polyamide epichlorohydrin to macromolecule weight ratio from 1:99 to 20:80, such as, e.g., 1:99 or 5:95 or 10:90 or 20:80.

In certain embodiments, the pre-reacted resin comprises a polymer having an epoxy to macromolecule weight ratio from 1:99 to 20:80, such as, e.g., 1:99 or 5:95 or 10:90 or 20:80.

The pre-reacted macromolecule is present in controlled release particles of the invention in an amount effective to improve the barrier properties and environmental biodegradability of the membrane. The amount of pre-reacted resin on a dry basis (weight of pre-reacted resin per weight of dry matter in the shell material) can be, e.g., from 1 wt. % or 5 wt. % or 10 wt. % or 20 wt. % to 40 wt. % or 70 wt. % or 84 wt. %.

In certain embodiments, the polyamide epichlorohydrin comprises a water-soluble polymeric reaction product of epichlorohydrin and a polyamide derived from a polyalkylene polyamine and a mixture of (a) a saturated aliphatic dibasic carboxylic acid containing from about 3 to about 10 carbon atoms and (b) a polymeric fat acid. As used herein, the polyamide epichlorohydrin may also comprise a reaction product between epichlorohydrin and a monomeric or polymeric amine such that the resulting reaction product has at least one cationic functional group. These adducts can be in the form of monomeric compounds (e.g., the reaction product of epichlorohydrin and ethylene diamine), or can be in polymeric form (e.g., the reaction product between epichlorohydrin, and polyamide-polyamines or polyethyleneimines). One type of amino compound which can be reacted with epichlorohydrin to form adducts useful in the present invention comprises monomeric di-, tri- and higher amines having primary or secondary amino groups in their structures. Examples of useful diamines of this type include bis-2-aminoethyl ether, N,N-dimethylethylenediamine, piperazine, and ethylenediamine. Examples of useful triamines of this type include N-aminoethyl piperazine, and dialkylene triamines such as diethylenetriamine, and dipropylenetriamine. Such amine materials are reacted with epichlorohydrin to form the cationic amino-epichlorohydrin adducts useful as crosslinking agents herein. Preparation of these adducts, as well as a more complete description of the adducts themselves, can be found in U.S. Pat. No. 4,310,593 to Gross, and in Ross et al., “Some reactions of epichlorohydrin with amines.” The Journal of Organic Chemistry 29, no. 4 (1964): 824-826.

In certain embodiments, the macromolecule in the pre-reacted resin comprises a protein. The protein preferably comprises a material selected from the group consisting of proteinogenic L-amino acids, animal or plant proteins, protein isolates, animal or plant protein hydrosylates, animal or plant proteins produced by physicochemical or fermentative or enzymatic treatment. Animal based proteins comprise those derived from meat (mammals, birds, reptiles, amphibians, fish), crabs, crustaceans, mussels, mollusks, insects, eggs, milk, casein, whey, gelatin, algae and mixtures thereof. Plant based proteins comprise those derived from cereals such as wheat, barley, rye, spelt gluten, rapeseed, sunflower, rice, potato, corn, soybean, bean, pea, chickpea, lentil, lupin, alfalfa, hemp, chitosan, and mixtures thereof.

The protein is present in particles of the invention in an amount effective to improve the barrier properties and environmental biodegradability of the membrane. The ratio of protein to polyamide epichlorohydrin can range from 99:1, or 95:5, or 90:10, or 80:20, or 70:30, to 50:50.

In certain embodiments, the macromolecule in the pre-reacted resin comprises a polysaccharide. The polysaccharide preferably comprises a member selected from the group consisting of natural starches such as tapioca, potato, corn, rice, wheat; modified starches such as carboxy modified polysaccharide or cellulose such as carboxymethyl starch, carboxymethyl chitosan, chitosan oligosaccharide, hydroxy propyl methyl starch, hydroxy propyl cellulose, ethyl cellulose, methyl cellulose, and octenyl succinic anhydride modified starch.

The polysaccharides are present in particles of the invention in an amount effective to improve the environmental biodegradability of the particles. The ratio of polysaccharide to polyamide epichlorohydrin can range from 99:1, or 95:5, or 90:10, or 80:20, or 70:30, to 50:50.

In certain embodiments, the polyphenol containing material preferably comprises a material selected from the group consisting of lignin, tannic acid, banana peel powder, sugar bagasse, quercetin, kaempferol, catechins, and anthocyanins, and flavonoids. Such polyphenol containing material may also comprise hemicelluloses, celluloses, and modified cellulose materials.

The polyphenol containing material present in particles of the invention in an amount effective to improve the barrier properties and environmental biodegradability of the membrane. The ratio of polyphenol containing material to polyamide epichlorohydrin can range from 99:1, or 95:5, or 90:10, or 80:20, or 70:30, to 50:50.

In certain embodiments, the inorganic solid particles comprise a member selected from the group consisting of organically modified or water insoluble clays, minerals, salts such as talc, calcium carbonate, bentonite, calcium chloride, hydroxyapatite, calcium phosphate, talc, kaolin, montmorrilonite, and amine modified kaolin.

The inorganic solid particles are present in particles of the invention in an amount effective to improve the barrier properties of the membrane. The weight ratio of inorganic solid particles to polyamide epichlorohydrin can range from 1:99, or 5:95, or 10:90, or 20:80, to 30:70.

The hydrophobic active ingredient is a hydrophobic substance that is active (or effective) to provide a desired effect, alone or in combination with other substances and/or conditions. It is present in the particles in an amount effective to provide a desired effect. The amount can be, e.g., from 47 wt. % or 59 wt. % or 66 wt. % to 73 wt. % or 78 wt. % or 81 wt. % or 93.5 wt. %, wherein the weight percentages are based on the weight of hydrophobic active divided by the weight of dry matter in the composition.

The hydrophobic active ingredient is preferably a member selected from the group consisting of a flavorant, a fragrance, a chromogen, a dye, an essential oil, a sweetener, an oil, a pigment, an active pharmaceutical ingredient, a moldicide, a herbicide, a fertilizer, a pheromone, phase change material, an adhesive, a vitamin oil, a vegetable oil, a triglyceride and a hydrocarbon.

Suitable flavorants include but are not limited to oils derived from plants and fruits such as citrus oils, fruit essences, peppermint oil, clove oil, oil of wintergreen, anise, lemon oil, apple essence, and the like. Artificial flavoring components are also contemplated. Those skilled in the art will recognize that natural and artificial flavoring agents may be combined in any sensorially acceptable blend. All such flavors and flavor blends are contemplated by this invention. Carriers may also be mixed with flavors to reduce the intensity, or better solubilize the materials. Carriers such as vegetable oils, hydrogenated oils, triethyl citrate, and the like are also contemplated by the invention.

Suitable fragrances include but are not limited to compositions comprising materials having an Log P (logarithm of octanol-water partition coefficient) of from about 2 to about 12, from about 2.5 to about 8, or even from about 2.5 to about 6 and a boiling point of less than about 280° C., from about 50° C. to about less than about 280° C., from about 50° C. to about less than about 265° C., or even from about 80° C. to about less than about 250° C.; and optionally, an ODT (odor detection threshold) of less than about 100 ppb, from about 0.00001 ppb to about less than about 100 ppb, from about 0.00001 ppb to about less than about 50 ppb or even from about 0.00001 ppb to about less than about 20 ppb. Diluents that are miscible in the fragrance oil, and act to reduce the volatility of the fragrance oil, such as isopropyl myristate, iso E super, triethyl citrate, vegetable oils, hydrogenated oils, neobee, and the like are also contemplated by the invention.

Suitable chromogens include but are not limited to Michler's hydrol, i.e. bis(p-dimethylaminophenyl)methanol, its ethers, for example the methyl ether of Michler's hydrol and the benzylether of Michler's hydrol, aromatic sulfonic and sulfinic esters of Michler's hydrol, for example the p-toluenesulfinate of Michler's hydrol, and derivatives of bis(p-dimethylaminophenyl)methylamine, e.g., N[bis(p-dimethylaminophenyl)methyl]morpholine.

Suitable dyes include but are not limited to Sudan Red 380, Sudan Blue 670, Baso Red 546, Baso Blue 688, Sudan Yellow 150, Baso Blue 645, Flexo Yellow 110, and Flexo Blue 630, all commercially available from BASF; Oil Red 235, commercially available from Passaic Color and Chemical; Morfast Yellow 101, commercially available from Morton; Nitro Fast Yellow B, commercially available from Sandoz; Macrolex Yellow 6G, commercially available from Mobay. Preferred dyes are those having good solubility in aromatic solvents.

Suitable essential oils include but are not limited to those obtained from thyme, lemongrass, citrus, anise, clove, aniseed, roses, lavender, citronella, eucalyptus, peppermint, camphor, sandalwood, cinnamon leaf and cedar. Essential oils that exhibit antimicrobial properties are also contemplated by this invention.

Suitable sweeteners include but are not limited to materials that contain varying amounts of disaccharide and/or fructose; erythritol, honey, and/or evaporated cane juice; and rebaudioside A, and the like.

Suitable pigments include but are not limited to pearl pigments of mica group such as titanium dioxide-coated mica and colored titanium dioxide-coated mica; and pearl pigments of bismuth oxychlorides such as colored bismuth oxychloride. Such pigments are available on the market under various trade names: Flamenco series (by the Mearl Corporation), TIVIIRON COLORS (by MERCK) as titanium dioxide-coated mica, Timica Luster Pigments (by MEARL). Cloisonee series (by MEARL), COLORON series (by MERCK), SPECTRA-PEARL PIGMENTS (by Mallinckrodt) as colored titanium dioxide-coated mica and MIBIRON COLORS series (by MERCK) as colored bismuth oxychloride.

Suitable active pharmaceutical ingredients include but are not limited to water insoluble materials that have a melting point below 50° C.

Suitable moldicides include but are not limited to an inorganic biocide selected from the group consisting of a metal, a metal compound and combinations thereof. Preferably, the inorganic biocide is copper, cobalt, boron, cadmium, nickel, tin, silver, zinc, lead bismuth, chromium and arsenic and compounds thereof. More preferably, the copper compound is selected from the group consisting of copper hydroxide, cupric oxide, cuprous oxide, copper carbonate, basic copper carbonate, copper oxychloride, copper 8-hydroxyquinolate, copper dimethyldithiocarbamate, copper omadine and copper borate. Suitable moldicides further include but are not limited to fungicidal compounds such as, e.g., isothiazolone compounds. Typical examples of isothiazolone compounds include but not limited to: methylisothiazolinone; 5-chloro-2-methyl-4-isothiazoline-3-one, 2-methyl-4-isothiazoline-3-one, 2-n-octyl-4-isothiazoline-3-one, 4,5-dichloro-2-n-octyl-4-isothiazoline-3-one, 2-ethyl-4-isothiazoline-3-one, 4,5-dichloro-2-cyclohexyl-4-isothiazoline-3-one, 5-chloro-2-ethyl-4-isothiazoline-3-one, 2-octyl-3-isothiazolone, 5-chloro-2-t-octyl-4-isothiazoline-3-one, 1,2-benzisothiazoline-3-one, preferably 5-chloro-2-methyl-4-isothiazoline-3-one, 2-methyl-4-isothiazoline-3-one, 2-n-octyl-4-isothiazoline-3-one, 4,5-dichloro-2-n-octyl-4-isothiazoline-3-one, 1,2-benzisothiazoline-3-one, etc., more preferably 5-chloro-2-methyl-4-isothiazoline-3-one, 2-n-octyl-4-isothiazoline-3-one, 4,5-dichloro-2-n-octyl-4-isothiazoline-3-one, 1,2-benzisothiazoline-3-one, chloromethyl-isothiazolinone, 4,5-Dichloro-2-n-octyl-3(2H)-isothiazolone and 1,2-benzisothiazolin-3-one.

Suitable herbicides include but are not limited to 2-(2-chloro-4-methylsulfonylbenzoyl)-1,3-cyclohexanedione, 2-(2-nitrobenzoyl)-4,4-dimethyl-1,3-cyclohexanedione, 2-(2-(nitrobenzoyl)-5,5-dimethyl-1,3-cyclohexanedione, and their 2-benzoylcyclohexanedione derivatives, in addition to those listed in WO2006024411A2.

Suitable phase change materials include but are not limited to a crystalline alkyl hydrocarbon which is comprised of one or more crystalline straight chain alkyl hydrocarbons having 14 or more carbon atoms and heats of fusion greater than 30 cal/g. The melting and freezing point of the alkyl hydrocarbon is in the range of 0° to 800° C., preferably 5° to 50° C., and most preferably, 18° to 330° C. Representative materials are crystalline polyolefins such as polyethylene, polypropylene, polybutene, crystalline polystyrene, crystalline chlorinated polyethylene and poly(4-methylpentene-1). Crystalline ethylene copolymers such as ethylene vinylacetate, crystalline ethylene acrylate copolymers, ionomers, crystalline ethylene-butene-1 copolymers and crystalline ethylene-propylene copolymers are also useful polyolefins. Preferably, the polyolefins are crosslinked such that they are form stable upon heating above their crystalline melting point.

Suitable adhesives include but are not limited to compositions comprising an elastomer and a tackifying agent. The elastomer adds toughness to the adhesive film and also is responsible for at least part of the required initial pressure-sensitive tackiness. The elastomeric materials are water insoluble and are inherently tacky or are capable of being rendered tacky by mixture with compatible tackifying resins. Preferably the elastomers are natural rubber or butadiene or isoprene synthetic polymers or copolymers such as butadiene-isobutylene copolymers, butadiene-acrylonitrile copolymers, butadiene-styrene copolymers, polychloroprene or similar elastomers. A combination of the above elastomers may be utilized. Preferred tackifying agents include unsaturated natural resins such as rosin or derivatives thereof, such as rosin esters of polyols such as glycerol or pentaerythritol, hydrogenated rosins or dehydrogenated rosins

Suitable vitamin oils include but are not limited to fat-soluble vitamin-active materials, pro vitamins and pure or substantially pure vitamins, both natural and synthetic, or chemical derivatives thereof, crude extractions containing such substances, vitamin A, vitamin D, and vitamin E active materials as well as vitamin K, carotene and the like, or mixtures of such materials. The oil-soluble vitamin oil concentrate may be a high potency fish liver oil containing vitamin A and/or D, a synthetic vitamin A palmitate and/or acetate concentrated in an oil solution, vitamin D, or D either concentrated in oil solution or as an oleaginous resin, vitamin E (d-alpha tocopheryl acetate) in an oil solution, or vitamin K in oil solution, or beta-carotene as a crystalline oil suspension in oil.

Suitable vegetable oils include but are not limited to oils derived from palm, corn, canola, sunflower, safflower, rapeseed, castor, olive, soybean, coconut and the like in both the unsaturated forms and hydrogenated forms, and mixtures thereof.

Suitable triglycerides include but are not limited to those disclosed in U.S. Pat. No. 6,248,909B1.

Suitable hydrocarbons that can be the active or can be used in combination with the active in order to change the physical or chemical properties of the active, include but are not limited to, waxes, density modifiers, surface tension modifiers, melting point modifiers, viscosity modifiers, and mixtures thereof. Examples include animal waxes such as beeswax, plant waxes such as carnauba wax, candelilla wax, bayberry wax, castor wax, tallow tree wax, soya wax, rice bran wax, hydrogenated rice bran wax, soya wax, hydrogenated soya wax, hydrogenated vegetable oil. Examples of petroleum derived waxes are paraffin waxes and microcrystalline waxes. An example of synthetic wax is polyethylene wax. Examples of materials that can modify the density of the active phase in the particle are brominated vegetable oil, nanoclays such as montmorrilonite or kaolin, hydrophobically modified clays, hydrophobically modified precipitated silicas or fumed silicas. Examples of oil thickening agents are waxes mentioned above, modified organopolysiloxanes, silicone gums, hydrogenated castor oil, paraffin oils, polyolefins, and the like.

Cationic particles have a higher probability of adhering to anionic fabric in the laundering environment. Amine-functionality containing materials that can be incorporated into the spray-ready emulsion, which may have a favorable effect on adhesion of particles onto skin, hair, or fabric substrates comprise a polymer selected from the group consisting of polysaccharides, in one aspect, cationically modified starch and/or cationically modified guar; polysiloxanes; poly diallyl dimethyl ammonium halides; copolymers of poly diallyl dimethyl ammonium chloride and polyvinyl pyrrolidone; a composition comprising polyethylene glycol and polyvinyl pyrrolidone; acrylamides; imidazoles; imidazolinium halides; polyvinyl amine; copolymers of poly vinyl amine and N-vinyl formamide; polyvinylformamide, copolymers of polyvinylamine and polvyinylalcohol oligimers of amines, in one aspect a diethylenetriamine, ethylene diamine, bis(3-aminopropyl)piperazine, N,N-Bis-(3-aminopropyl)methylamine, tris(2-aminoethyl)amine and mixtures thereof; polyethyleneimime, a derivatized polyethyleneimine, in one aspect an ethoxylated polyethyleneimine; diester quaternary ammonium surfactants such as methyl bis-[ethyl(coconut)]-2-hydroxyethyl ammonium methyl sulfate, methyl bis-[ethyl(decyl)]-2-hydroxyethyl ammonium methyl sulfate, methyl bis-[ethyl(dodeceyl)]-2-hydroxyethyl ammonium methyl sulfate, methyl bis-[ethyl(lauryl)]-2-hydroxyethyl ammonium methyl sulfate, methyl bis-[ethyl(palmityl)]-2-hydroxyethyl ammonium methyl sulfate, methyl bis-[ethyl(soft-tallow)]-2-hydroxyethyl ammonium methyl sulfate, and the like; diester quat combined with laminate nanoclays such as laponite, bentonite, montmorillonite, and the like; chitosan with various degrees of deacetylation, carboxymethyl chitosans, glycol chitosans, whey protein, sodium caseinate, silk protein, 1H-Imidazolium, 1-ethenyl-3-methyl-, chloride, polymer with 1-ethenyl-2-pyrrolidinone, polyamines, polysaccharides with cationic modification, and mixtures thereof. Polysaccharides can be employed with cationic modification and alkoxy-cationic modifications, such as cationic hydroxyethyl, cationic hydroxy propyl. For example, cationic reagents of choice are 3-chloro-2-hydroxypropyl trimethylammonium chloride or its epoxy version. Furthermore, up to 5 different types of functional groups may be attached to the polysaccharides. Also, polymer graft chains may be differently modified than the backbone. The counterions can be any halide ion or organic counter ion. The preferred cationic starch has a molecular weight of from about 100,000 to about 500,000,000, preferably from about 200,000 to about 10,000,000 and most preferably from about 250,000 to about 5,000,000. The preferred cationic starch products are HI-CAT CWS42 and HI-CAT 02 and are commercially available from ROQUETTE AMERICA, Inc. The preferred cationic guar has a molecular weight of from about 50,000 to about 5,000,000. The preferred cationic guar products are Jaguar C-162 and Jaguar C-17 and are commercially available from Rhodia Inc.

The deposition aid is present in the controlled release particles in an amount on a dry basis (weight of deposition aid per weight of dry matter in the suspension) from 0.5 wt. % or 1 wt. % or 1.5 wt. % or 3.5 wt. % to 5 wt. % or 7 wt. % of the weight of the particle.

The controlled release particles are preferably spherical but non-spherical shapes are also within the scope of the invention. The particles preferably have a diameter from 0.05-250 microns, or from 0.1 microns to less than 100 microns.

Method of Making the Particles

The controlled release particles of the invention preferably comprise a core and a polymeric shell, wherein the core comprises: (1) at least one hydrophobic active ingredient; and (2) at least one pre-reacted resin; and the polymeric shell is obtained by any one of: (a) condensation reactions, (b) free radical polymerization reactions, (c) interfacial polymerization reactions, or (d) coacervation of pre-formed polymers followed by crosslinking of the thereby obtained coacervates by using a crosslinker.

In general, aminoplast capsules are made by condensing methylolated amine resin onto the oil droplets in an oil-in-water emulsion. Increasing the temperature crosslinks the polymers to make a low permeability membrane. Polyurea capsules made via interfacial polymerization achieves a shell at the oil-water interface via a chemical reaction between isocyanates and other monomers dissolved in the oil phase with amines dissolved in the water phase. The choice of monomers, crosslinking conditions, and emulsifiers used influences the permeability properties of the membrane. Polyacrylate capsules are made by free radical polymerization of polar acrylates dissolved in the oil phase. Polymerization of acrylates increases the molecular weight of the monomers, and such acrylate polymers become insoluble in both the oil phase and water phase, and establish themselves at the oil-water interface. The choice of emulsifier, type of acrylate monomers, initiators, and reaction conditions influences the permeability of the membrane. Complex coacervation capsules are made by emulsifying the oil phase in a solution of one polyelectrolyte (e.g., gelatin). Next, another polyelectrolyte (e.g., gum Arabic) is added to the suspension. Upon adjustment of solution pH, one of the polyelectrolytes adopts a charge that is opposite to the other polyelectrolyte. Such a change induces coacervation, or a polyelectrolyte interaction, that has low solubility in water. Such coacervate deposits onto the dispersed oil droplets and the coacervate can be crosslinked with an aldehyde to permanently “fix” the coacervate into place (such that the coacervate does not break down upon further dilution of the suspension).

In certain embodiments, the aldehyde comprises a member selected from the group consisting of aliphatic dialdehydes, aromatic dialdehydes, cyclic dialdehydes, and polyaldehydes. Nonlimiting examples of one or more aldehydes include, but are not limited to, valeraldehyde, capronaldehyde, caprylaldehyde, decanal, succinic dialdehyde, cyclohexanecarbaldehyde, cyclopentanecarbaldehyde, 2-methyl-1-propanal, 2-methylpropionaldehyde, acetaldehyde, acrolein, aldosterone, antimycin A, 8′-apo-p-caroten-8′-al, benzaldehyde, butanal, chloral, citral, citronellal, crotonaldehyde, dimethylaminobenzaldehyde, folinic acid, fosmidomycin, furfural, glutaraldehyde, glutardialdehyde, glyceraldehyde, glycolaldehyde, glyoxal, glyoxylic acid, heptanal, 2-hydroxybenzaldehyde, 3-hydroxybutanal, hydroxymethylfurfural, 4-hydroxynonenal, isobutanal, isobutyraldehyde, methacrolein, 2-methylundecanal, mucochloric acid, N-methylformamide, 2-nitrobenzaldehyde, nonanal, octanal, oleocanthal, orlistat, pentanal, phenylethanal, phycocyanin, piperonal, propanal, propenal, protocatechualdehyde, retinal, salicylaldehyde, secologanin, streptomycin, strophanthidin, tylosin, vanillin, cinnamaldehyde glutaraldehyde, glyoxal, dialdehyde starch, polyethylene glycol dialdehyde, succinaldehyde, 1,3-propane dialdehyde, 1,4-butane dialdehyde, 1,5-pentane dialdehyde, dialdehyde starch, dialdehyde chitosan, reduced sugars containing aldehyde moieties, and the mixtures thereof.

The processes described in the references cited above can be used to prepare microcapsules, with one procedural change. The pre-reacted resin of the invention is incorporated into the oil phase prior to emulsification.

In certain embodiments, a plasticizer is included in the oil phase and is at least one member selected from the group consisting of methyl esters of rosin, polyazelate esters, di-fatty acid esters, citrate esters, polyadipate esters and polyester resins consisting of inner and intra-esters of polyhydroxy carboxylic acids.

The inventors have discovered that pursuing a high degree of crosslinking in making microcapsules via chemical reaction processes that comprise interfacial polymerization, polycondensation reactions, addition reactions, free radial polymerization reactions, and the like, may provide a membrane with good barrier properties and mechanical properties; however, such membranes have poor environmental biodegradability. Not to be limited by theory, a high degree of crosslinking results in the absence of both functional groups and flexibility that hinders the ability of microbes to form a biofilm around the polymer membrane followed by digestion of membrane to improve biodegradability. Incorporation of biodegradable materials into the membrane via the use of pre-reacted resin can improve barrier properties of the membrane (by providing a more tortuous path for the encapsulated material to diffuse, poor miscibility of the encapsulated active material in the polymer, biodegradable polymer segments swell with water reducing the diffusion of the encapsulated active), and improved environmental biodegradability of the membrane due to the presence of amino acids, glucose units, esters, amides, and other functional groups whose breakdown is enabled by enzymes that the microbes readily secrete.

In certain embodiments of providing a powder composition of the invention, or making the dehydrated forms of the pre-reacted resin, spray drying is an economical process that can be used. Spray drying of the particle suspension is preferably conducted in a co-current spray dryer, at an inlet air temperature of 325 to 415° F. (163-213° C.), preferably from 355 to 385° F. (179-196° C.) and an outlet air temperature of 160 to 215° F. (71-101° C.), preferably from 175-195° F. (79-91° C.).

Compositions Containing the Particles

The invention further comprises compositions (e.g., products, articles of manufacture, etc.) comprising the controlled release particles. Such compositions include but are not limited baby care, beauty care, fabric & home care, family care, feminine care, health care, snack and/or beverage products or devices intended to be used or consumed in the form as sold, and not intended for subsequent commercial manufacture or modification. Such products include but are not limited to fine fragrances (e.g., perfumes, colognes eau de toilettes, after-shave lotions, pre-shave, face waters, tonics, and other fragrance-containing compositions for application directly to the skin), diapers, bibs, wipes; products for and/or methods relating to treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use; and shaving products, products for and/or methods relating to treating fabrics, hard surfaces and any other surfaces in the area of fabric and home care, including: air care, car care, dishwashing, fabric conditioning (including softening), laundry detergency, laundry and rinse additive and/or care, hard surface cleaning and/or treatment, and other cleaning for consumer or institutional use; products and/or methods relating to bath tissue, facial tissue, paper handkerchiefs, and/or paper towels; tampons, feminine napkins; products and/or methods relating to oral care including toothpastes, tooth gels, tooth rinses, denture adhesives, tooth whitening; over-the-counter health care including cough and cold remedies, pain relievers, RX pharmaceuticals, pet health and nutrition, and water purification; processed food products intended primarily for consumption between customary meals or as a meal accompaniment (non-limiting examples include potato chips, tortilla chips, popcorn, pretzels, corn chips, cereal bars, vegetable chips or crisps, snack mixes, party mixes, multigrain chips, snack crackers, cheese snacks, pork rinds, corn snacks, pellet snacks, extruded snacks and bagel chips); and coffee. Moreover, such products include, but are not limited to, a powdered food product, a fluid food product, a powdered nutritional supplement, a fluid nutritional supplement, a fluid fabric enhancer, a solid fabric enhancer, a fluid shampoo, a solid shampoo, hair conditioner, body wash, solid antiperspirant, fluid antiperspirant, solid deodorant, fluid deodorant, fluid detergent, solid detergent, fluid hard surface cleaner, solid hard surface cleaner, a fluid fabric refresher spray, a diaper, an air freshening product, a nutraceutical supplement, a controlled release fertilizer, a controlled release insecticide, a controlled release dye, and a unit dose detergent comprising a detergent and the controlled release particles in a water soluble film.

Fluid compositions of the invention preferably further comprise at least one suspension agent to suspend the controlled release particles, wherein the at least one suspension agent is at least one member selected from the group consisting of a rheology modifier, a structurant and a thickener. The at least one suspension agent preferably has a high shear viscosity at, 20 sec⁻¹ shear rate and at 21° C., of from 1 to 7000 cps and a low shear viscosity, at 0.5 sec⁻¹ shear rate and at 21° C., of greater than 1000 cps or 1000-200,000 cps. In certain embodiments, the composition has a high shear viscosity, at 20 sec⁻¹ and at 21° C., of from 50 to 3000 cps and a low shear viscosity, at 0.5 sec⁻¹ shear rate and at 21° C., of greater than 1000 cps or 1000-200,000 cps.

Preferably, the at least one suspension agent is selected from the group consisting of polyacrylates, polymethacrylates, polycarboxylates, pectin, alginate, gum arabic, carrageenan, gellan gum, xanthan gum, guar gum, gellan gum, hydroxyl-containing fatty acids, hydroxyl-containing fatty esters, hydroxyl-containing fatty waxes, castor oil, castor oil derivatives, hydrogenated castor oil derivatives, hydrogenated castor wax and mixtures thereof.

The invention further encompasses a slurry comprising particles of the invention. Said slurry may be combined with an adjunct ingredient to form a composition, for example, a consumer product. In certain embodiments, the slurry comprises at least one processing aid selected from the group consisting of water, aggregate inhibiting materials such as divalent salts, particle suspending polymers, and mixtures thereof. Examples of aggregate inhibiting materials include salts that can have a charge shielding effect around the particle, such as, e.g., magnesium chloride, calcium chloride, magnesium bromide, magnesium sulfate and mixtures thereof. Examples of particle suspending polymers include polymers such as xanthan gum, carrageenan gum, guar gum, shellac, alginates, chitosan; cellulosic materials such as carboxymethyl cellulose, hydroxypropyl methyl cellulose and cationically charged cellulosic materials; polyacrylic acid; polyvinyl alcohol; hydrogenated castor oil; ethylene glycol distearate; and mixtures thereof.

In certain embodiments, the slurry comprises at least one carrier selected from the group consisting of polar solvents, including but not limited to, water, ethylene glycol, propylene glycol, polyethylene glycol, glycerol, non-polar solvents including but not limited to mineral oil, perfume raw materials, silicone oils, hydrocarbon paraffin oils, and mixtures thereof.

In certain embodiments, a perfume oil is combined with the slurry comprising microcapsules to provide multiple benefits. The emulsified perfume oil will increase the viscosity of the slurry and prevent the phase separation of the microcapsule particles. The mixture provides a way to deliver non-encapsulated and encapsulated fragrance from the same slurry.

In certain embodiments, the composition has at least two controlled release technologies, which release different hydrophobic oil compositions and are selected from the group consisting of neat oils, friction-triggered release microcapsules and water-triggered release microcapsules.

The invention will be illustrated in more detail with reference to the following Examples, but it should be understood that the present invention is not deemed to be limited thereto.

### EXAMPLES

Materials and Methods

The following is a representative perfume oil composition used for capsule making.

TABLE 1
Perfume oil composition

| Material | wt. % | Functionality |
|---|---|---|
| CITRONELLYL NITRILE | 1.00% | NITRILE |
| TRIPLAL | 0.25% | ALDEHYDE |
| FLORHYDRAL | 0.10% | ALDEHYDE |
| ALDEHYDE C-10 | 0.10% | ALDEHYDE |
| ALDEHYDE C-12 LAURIC | 0.20% | ALDEHYDE |
| ALLYL CYCLOHEXYL PROPIONATE | 1.00% | ESTER |
| CETALOX | 0.20% | FURAN |
| ANISIC ALDEHYDE | 0.10% | ALDEHYDE |
| CYCLACET | 10.00% | ESTER |
| CYCLAPROP | 5.00% | ESTER |
| DIHYDROMYRCENOL | 10.00% | ALCOHOL |
| DIPHENYL OXIDE | 1.00% | OXIDE |
| HABANOLIDE | 2.50% | KETONE |
| YARA YARA | 2.00% | ETHER |
| CIS-3-HEXENYL SALICYLATE | 2.00% | ESTER |
| VERDOX | 2.50% | ESTER |
| HEXYLCINNAMIC ALDEHYDE | 20.00% | ALDEHYDE |
| BHT | 0.50% | 0.0025 |
| ISO E SUPER | 2.50% | KETONE |
| KOAVONE | 2.50% | 0.0625 |
| EUCALYPTOL | 0.20% | ALCOHOL |
| MANZANATE, 10% IPM | 0.50% | ESTER |
| MUSCENONE, 10% IPM | 0.50% | KETONE |
| LAEVO CARVONE, 10% IPM | 0.50% | 0.0025 |
| METHYL ANTHRANILATE | 0.10% | ESTER |
| METHYL IONONE GAMMA | 1.25% | KETONE |
| LILIAL | 10.00% | ALDEHYDE |
| ALDEHYDE C-12 MNA, 10% DPG | 0.50% | ALDEHYDE |
| MYRAC ALDEHYDE | 0.50% | ALDEHYDE |
| D-LIMONENE | 5.00% | TERPENE |
| PEONILE | 2.50% | NITRILE |
| ETHYLENE BRASSYLATE | 12.50% | ESTER |
| PHENOXANOL | 2.50% | ALCOHOL |

Scanning Electron Microscopy

A Phenom Pure (Nanoscience Instruments Model PW-100-019) Scanning Electron Microscope is used to understand the particle morphology, and nature of particle deposits on fabrics. PELCO tabs carbon tape (12 mm OD, Ted Pella product number 16084-1) is applied to an aluminum specimen mount (Ted Pella Product No 16111). Next, the powder sample is placed onto the carbon tape using a transfer spatula. Excess powder is removed by blowing Dust-Off compressed gas onto the sample. The stub is then left in a desiccator under vacuum for 16 hours to flash off any volatiles. The sample is then placed into the Phenom Pure, and imaged to visualize particle morphology.

Detergent/Water Dissolution+Fabric Preparation

To 9.75 grams of a detergent solution (1 gram of liquid detergent added to 99 grams of water, then filtered through Whatman 597 filter catalog number 10311808) is added powder or slurry that achieves a concentration of approximately 1 wt. % perfume oil in the detergent solution. For water solubility, the powder is simply dosed into water rather than detergent solution. For the Detergent Dissolution Test, the sample is mixed at 200 RPM for 30 minutes at 33.3° C. A pre-weighed 3 inch diameter circle of black 100% cotton fabric is placed in a Buchner funnel attached to a vacuum line. 2 mL of the solution is then poured through the fabric, followed by a wash of 2 mL water. The fabric is allowed to air dry overnight.

Odor Evaluation

There are two techniques utilized to evaluate odor of fabrics:

1) The dried fabrics from the Detergent Dissolution Test+Fabric Preparation test are evaluated olfactive by a panel before and after rubbing. A subjective grading scale is used to grade fabrics before rubbing and after rubbing. In the case of before rubbing, the control that is used is a fabric treated with neat fragrance oil in the detergent solution. In the case of rubbed fabric, the control is the fabric before rubbing is performed.

TABLE 2
Odor grading scale

| Odor Grade | Description |
|---|---|
| 0 | No Difference vs. Control |
| 1 | Slight Difference vs. Control |
| 2 | Noticeable Difference vs. Control (detectable difference) |
| 3 | Significant difference vs. control (high intensity vs. control) |
| 4 | Very High Intensity Bloom vs. control |
| 5 | Extremely High Intensity vs. Control |

The dried fabrics from the Detergent Dissolution Test+Fabric Preparation test are evaluated by an Odor Meter (Shinyei Technology model OMX-SRM) before and after rubbing. This method reports the total concentration of volatiles in the headspace and is reported in milligrams per cubic meter as a function of time

Leakage Stability

A suspension of microcapsules is incorporated into Liquid Fabric Softener to deliver approximately 0.5 wt. % perfume usage level in the fabric softener. The mixture is aged 4 weeks at 40° C. in sealed glass jars. After aging, approximately 1.6 grams of the fabric softener mixture is diluted with 10 grams of water. 10 mL of isooctane is added to the vial, and the vial is inverted 10 times. 2-4 grams of sodium chloride is added to achieve a better separation. The sample is placed on a platform shaker for 10 minutes at 225-235 RPM agitation. After mixing, the sample is centrifuged at 2800 RPM for 2 minutes. Approximately 5 mL of the isooctane layer is removed from the vial and filtered through a 0.45 micron syringe filter. 980 microliters of this filtrate is mixed with 20 microliters of internal standard in a 2 mL GC autosampler vial. The sample is analyzed by Gas Chromatography. GC conditions are shown in Table 3 below.

TABLE 3
GC CONDITIONS
Gas Chromatography/Mass Spectrophotometer Conditions

| Capillary Column | DB-5MS, 30 meter, 0.25 μm film, | |
|---|---|---|
| | ID = 0.25 mm | |
| Carrier Gas | UHP Helium, 1.2 mL/min through | |
| | the column | |
| Injection Volume | 1.0 μL, Split, Split Ratio 8.0:1 | |
| Injector Port Temperature | 250° | C. |

Oven Conditions

| | | |
|---|---|---|
| Initial Temperature | 40° | C. |
| Hold Time | 2 | minutes |
| Ramp | 5° | C./min |
| Final Temperature | 270° | C. |
| Final Hold | 6 | minutes |
| Total Run Time | 54.0 | minutes |

Mass Spectrophotometer Detector Conditions

| MS Source Temperature | 230° | C. |
|---|---|---|
| MS Quad | 150° | C. |
| Back Detector | 270° | C. |
| Tune File | Atune.u | |
| Scan Range | 40 to 600 amu | |
| Solvent Delay | 4.5 | minutes |

Calculate the average Response Factor of total area sum from standard calibration curve. See equation below:

RF=((Ax)*(Cis)/((Ais)(Cx))
where:
AX=Area of the compound
CX=Standard Concentration (mg)
Ais=Area of the internal standard
Cis=Internal Standard Concentration (mg)

Calculate sample concentration (mg). See equation below:

CXs=(((Ax)*(Cis)/((Ais)(RFAVE)))*df
where:
AX=Area of the compound
CXs=Sample Concentration (mg)
Ais=Area of the internal standard
RFAVE=Average Response Factor
df=Sample Dilution

By using the sample concentration (mg) of perfume oil found in the isooctane extract and dividing by the theoretical perfume dosed into the fabric softener, one can calculate the amount of perfume that has leaked out of the microcapsule during aging.

Biodegradability Test Method

Biodegradability testing is carried out according to protocol OECD 301D. The microcapsule membrane is isolated by going through the following steps: (1) Lyophilize the microcapsule slurry sample, (2) Methanol/toluene extraction of the lyophilized solids to assure less than 5% residual oil, (3) filtration of the solvent and extracted material, (4) vacuum drying at 60° C. and 0.3 torr for 24 hours, (5) water extraction of the vacuum dried powder to remove any water soluble components in the membrane, followed by filtration to recover the particles, (6) vacuum dry the powder to remove residual water at 0.3 torr for 1 day 60° C. The isolated polymer is then subjected to OECD 301D protocol, available at https://www.oecd.org/chemicalsafety/risk-assessment/1948209.pdf, with the following experimental conditions:
1) test substance concentration in the mineral medium is 5 mg/L.
2) 300 mL Biological Oxygen Demand (BOD) bottles with glass stoppers are used.
3) An incubator at 20° C. is used to age the samples in the dark.
4) The mineral stock solutions as provided in the method are prepared.
5) After letting the secondary effluent settle for at least 1 hour, a 10× (10 mL of secondary effluent is added to 90 mL of deionized water) secondary effluent is prepared with BOD water to make 100 mL total inoculum in a beaker. Then 0.5 mL of the 10× inoculum is added to each BOD bottle.
6) COD of the isolated polymer is measured using Hach kit.

The bottles are checked for dissolved oxygen at 0 days, 7, 14, 28, and 60 days. The percent degradation is analyzed via the calculations taught in the OECD 301D method.

### Example 1—Pre-Reacted Resin

1A: 150 grams of Gelatin high bloom strength is dissolved in 1350 grams of water at 40° C. 150 grams of a 20 wt. % solution of phenol is added to the solution and 19 grams of inorganic solid is dispersed in the suspension. Approximately 70 grams of a 20% solution of sodium hydroxide is added to adjust the pH to 9.5. The suspension is heated at 60° C. for approximately 4 hours. Approximately 22 grams of polyamine epichlorohydrin (Solenis) is added to yield a suspension. The homogeneous suspension is spray dried in a Bowen 3 ft diameter co-current spray drying tower using a 2-fluid nozzle at 70 psi air pressure, an inlet air temperature of 350° F. (177° C.) and an outlet temperature of 185° F. (85° C.). Dry powder with a median size of 19 microns is collected from the spray dryer. The powder is then heated at 110° C. for 60 minutes in an oven.

1B-150 grams of Casein (Naked Casein, amazon.com) is dissolved in 2850 grams of distilled water at room temperature. Approximately 22.5 grams of 10 wt. % sodium carbonate is added to achieve a pH of 8.0 150 grams of a 20 wt. % solution of phenol is added. Approximately 70 grams of a 20% solution of sodium hydroxide is added to the solution to adjust the pH to 9.5. The contents are heated at 60° C. for 4 hours. Next, approximately 22 grams of polyamine epichlorohydrin (Solenis) is added to the suspension. The homogeneous suspension is spray dried in a Bowen 3 ft diameter co-current spray drying tower using a 2-fluid nozzle at 70 psi air pressure, an inlet air temperature of 385° F. (196° C.) and an outlet temperature of 185° F. (85° C.). Dry powder with a median size of 19 microns is collected from the spray dryer. The powder is then heated at 110° C. for 30 minutes in an oven.

1C: 150 grams of pea protein (green boy group) is dissolved in 2850 grams of distilled water at room temperature. Approximately 6 grams of 20 wt. % sodium hydroxide is added to achieve a pH of 9.5. 150 grams of a 20 wt. % solution of phenol is added. Approximately 35 grams of a 20% solution of sodium hydroxide is added to the solution to adjust the pH to 9.5. The contents are heated at 60° C. for 4 hours. Next, approximately 21 grams of polyamine epichlorohydrin (Solenis) is added to the suspension. The homogeneous suspension is spray dried in a Bowen 3 ft diameter co-current spray drying tower using a 2-fluid nozzle at 70 psi air pressure, an inlet air temperature of 385° F. (196° C.) and an outlet temperature of 185° F. (85° C.). Dry powder with a median size of 19 microns is collected from the spray dryer. The powder is then heated at 110° C. for 30 minutes in an oven.

1D: 150 grams of Gelatin high bloom strength is dissolved in 1350 grams of water at 40° C. 31 grams of glycerin is added to the solution. Approximately 8.5 grams of a 20% solution of sodium carbonate is added to adjust the pH to 10.2. Approximately 10.5 grams of polyamine epichlorohydrin (Solenis) is added to yield a homogeneous solution. The homogeneous suspension is spray dried in a Bowen 3 ft diameter co-current spray drying tower using a 2-fluid nozzle at 70 psi air pressure, an inlet air temperature of 350° F. (177° C.) and an outlet temperature of 185° F. (85° C.). Dry powder with a median size of 19 microns is collected from the spray dryer. The powder is then heated at 110° C. for 60 minutes in an oven.

1E: 150 grams of Gelatin high bloom strength is dissolved in 1350 grams of water at 40° C. 150 grams of a 20 wt. % solution of phenol is added to the solution and 19 grams of inorganic solid is dispersed in the suspension. Approximately 70 grams of a 20% solution of sodium hydroxide is added to adjust the pH to 9.5. The suspension is heated at 60° C. for approximately 4 hours. Approximately 10.5 grams of polyamine epichlorohydrin (Solenis) is added to yield a suspension. The homogeneous suspension is spray dried in a Bowen 3 ft diameter co-current spray drying tower using a 2-fluid nozzle at 70 psi air pressure, an inlet air temperature of 350° F. (177° C.) and an outlet temperature of 185° F. (85° C.). Dry powder with a median size of 19 microns is collected from the spray dryer. The powder is then heated at 110° C. for 60 minutes in an oven.

1F: 150 grams of Gelatin high bloom strength is dissolved in 1500 grams of water at 40° C. 9 milliliters of 20% sodium hydroxide is added to adjust the pH of the gelatin solution. Next, 30 grams of casein is added to the batch and mixed for 15 minutes. Next, 150 grams of a 20 wt. % solution of phenol is added to the solution and 19 grams of inorganic solid is dispersed in the suspension. The suspension is heated at 60° C. for approximately 4 hours. Approximately 70 grams of a 20% solution of sodium hydroxide is added before and during the heating step to adjust the pH to 9.5. Approximately 25.5 grams of polyamine epichlorohydrin (Solenis) is added to yield a suspension. The homogeneous suspension is spray dried in a Bowen 3 ft diameter co-current spray drying tower using a 2-fluid nozzle at 70 psi air pressure, an inlet air temperature of 350° F. (177° C.) and an outlet temperature of 185° F. (85° C.). Dry powder with a median size of 19 microns is collected from the spray dryer. The powder is then heated at 110° C. for 60 minutes in an oven.

### Example 2—Complex Coacervate Capsule

Prepare the following solutions: 20 grams of Gelatin (bloom 300) in 600 grams of DI water. Heat the DI water to 70° C. (2 minutes in microwave). Add gelatin slowly. 20 grams of gum arabic in 400 g of DI water at room temperature, then heat to 40° C. Keep both solutions in 60° C. water bath.

Preparation of capsules
1) Calibrate the pH meter.
2) Preweigh 25 grams of perfume oil.
3) Preweigh 200 g of gum Arabic.
4) Preweigh 300 grams of gelatin solution that was prepared.
5) Add the perfume oil to the gelatin solution. Ultra turrax T-25 at 6500 RPM (lowest setting) using the fine T-25 assembly, for 20 seconds resulted in particles that were 10-30 microns in size.
6) Transfer the beaker to a magnetic stirrer, 150 RPM (very slow agitation). Check the pH. Should be 4.7-4.8. Add 1N HCl to adjust the pH within this range.
7) Add gum arabic solution slowly to the gelatin solution containing the perfume oil
8) Leave the contents on the magnetic stirrer for at least 3 hours (the solution should be at room temperature). Assure that the pH is between 4.7 and 4.8.
9) Add 0.050 grams of 25 wt. % glutaraldehyde to crosslink.

### Example 3—Complex Coacervate Capsules with Pre-Reacted Natural Polymer Resin

Prepare the following solutions: 20 grams of Gelatin (bloom 300) in 600 grams of DI water. Heat the DI water to 70° C. (2 minutes in microwave). Add gelatin slowly. 20 grams of gum arabic in 400 g of DI water at room temperature, then heat to 40° C. Keep both solutions in 60° C. water bath.

Preparation of capsules
1) Calibrate the pH meter.
2) Preweigh 25 grams of perfume oil and disperse 5 grams of the pre-reacted resin 1A by high shear mixing.
3) Preweigh 200 g of gum Arabic.
4) Preweigh 300 grams of gelatin solution that was prepared.
5) Add the perfume oil to the gelatin solution. Ultra turrax T-25 at 6500 RPM (lowest setting) using the fine T-25 assembly, for 20 seconds resulted in particles that were 10-30 microns in size.
6) Transfer the beaker to a magnetic stirrer, 150 RPM (very slow agitation). Check the pH. Should be 4.7-4.8. Add 1N HCl to adjust the pH within this range.
7) Add gum arabic solution slowly to the gelatin solution containing the perfume oil.
8) Leave the contents on the magnetic stirrer for at least 3 hours (the solution should be at room temperature). Assure that the pH is between 4.7 and 4.8.
9) Add 0.050 grams of 25 wt. % glutaraldehyde to crosslink.

### Example 4—Interfacial Polymerization Capsule

Prepare a premix of 40 grams perfume oil, 2.9 grams of epoxidized alcohol, 2.1 grams of cyanurate, 3.2 grams of aliphatic diisocyanate, 0.8 grams of trimethyl propane diglycidyl ether, 4 grams of pre-reacted resin of Example 1A, 1.5 grams of plasticizer. Mix for 15 minutes to obtain a homogeneous dispersion. Emulsify said dispersion in 200 milliliters of a 10 wt. % solution of gelatin at 40° C. to achieve a particle size of 25-35 microns. Adjust the pH of the suspension by adding 17.6 milliliters of 20% sodium carbonate. Heat the contents to 60° C. and maintain there for 1 hour. Add 1 gram of polyphenol, mix for 3 hours at 60° C. The finished microcapsules have a median particle size of 32 microns.

### Example 5—Leakage Stability and Performance Testing

Microcapsules slurries are formulated into liquid fabric softener (Downy Free & Clear), to deliver approximately 0.5 wt. % fragrance usage level in the liquid suspension, via the microcapsules or neat perfume oil. These samples are used for leakage stability testing and performance testing. The prepared mixtures are aged for 1 week at 40° C. After ageing, several tests are performed to evaluate the behavior of the capsules:
1) Optical microscopy to observe capsule deflation; and
2) Approximately 5 grams of the aged mixture is diluted with 5 grams of water to yield a dilute detergent solution containing approximately 0.25 wt. % fragrance oil. This diluted suspension is mixed for 30 minutes at a temperature of 25° C. at 250 RPM using a magnetic stirrer. Next, approximately 2 mL of the mixed solution is filtered through a black fabric, and allowed to dry overnight. The fabric odor intensity before rubbing and after rubbing is noted.

TABLE 4
Fabric odor performance of microcapsule slurries aged
in liquid fabric softener for 4 week at 40° C.

| | | Odor Grade | |
|---|---|---|---|
| | | Pre-Rub/ | Leakage |
| ID | Description of Capsule | Post-Rub | 4 wk/40° C. |
| Example 4A | Example 2 Capsules | 0/0 | 97% |
| Example 4B | Example 3 Capsules | 0/3 | 57% |
| Example 4C | Example 4 Capsules | 1.5/3 | 9% |

The incorporation of pre-reacted resin into complex coacervate capsule making, and interfacial polymerization capsule making significantly reduces the leakage of perfume oil out of the microcapsule when aged in a liquid fabric softener.

Environmental Biodegradability

| Pre-Rub or Post-Rub Odor Grade | Description |
|---|---|
| 0 | No odor |
| 1 | Slight odor |
| 2 | Noticeable odor |
| 3 | Highly Noticeable, obvious odor |
| 4 | Strong and highly impactful odor |

Microcapsules of various examples above were evaluated for environmental biodegradability by adapting the OCDE/OECD 301D Closed Bottle Test method, as described in the Biodegradability test method description.

Microcapsule suspensions were lyophilized, then extracted with methanol/toluene to remove the encapsulated perfume oil (12:1 ratio), and filtered. After vacuum drying, the powder was extracted with water (15:1 ratio) and filtered. Te powder was vacuum dried, ground into a fine powder using a mortar/pestle, and submitted for biodegradability testing.

TABLE 5
Mineral Oil Solutions

| Mineral | | | |
|---|---|---|---|
| Solution ID | Ingredient | Formula | Mass (g) |
| A | Potassium dihydrogen | KH₂PO₄ | 8.50 |
| | orthophosphate | | |
| | Dipostassium hydrogen | K₂HPO₄ | 21.75 |
| | orthophosphate | | |
| | Disodium hydrogen | Na₂HPO₄—2H₂O | 33.40 |
| | orthophosphate | | |
| | dehydrate | | |
| | Ammonium chloride | NH₄Cl | 0.50 |
| | Dissolve in water and | | |
| | bring to 1 L. pH to 7.4 | | |
| B | Calcium Chloride | CaCl₂ | 27.50 |
| | anhydrous | | |
| | OR | | |
| | Calcium Chloride | CaCl₂—2H₂O | 36.40 |
| | dehydrate | | |
| | Dissolve in water and | | |
| | bring to 1 L. | | |
| C | Magnesium sulfate | MgSO₄—7H₂O | 22.50 |
| | heptahydrate | | |
| | Dissolve in water and | | |
| | bring to 1 L. | | |
| D | Iron (III) chloride | FeCl₃—6H₂O | 0.25 |
| | hexahydrate | | |
| | Dissolve in water and | | |
| | bring to 1 L. | | |

Prepare approximately 300 mL solutions containing the particles to be tested (approximately 1.5 milligrams of the isolated polymer is added to each BOD bottle). Fill BOD bottles (300 mL capacity) just past the neck of the bottle. Insert stopper. Store BOD bottles in the dark in an incubator maintained at 20° C. Use dissolved oxygen meter (YSI 5000), and YSI5905 Dissolved Oxygen meter probe to measure oxygen at specific time points.

The dissolved oxygen measured values as a function of time, and the calculation methods presented in OECD 301D method are utilized to calculate the % biodegradability. The Environmental Biodegradability index is calculated by multiplying the measured % biodegradability by 100. The results are listed in Table 6 below.

TABLE 6
Environmental Biodegradability Results

| | OECD 301D % | Biodegradability |
|---|---|---|
| Material/Attribute | Biodegradability (28 day) | Index |
| Example 2 | 65% | 65 |
| Example 3 | 62% | 62 |
| Example 4 | 61% | 61 |

A biodegradability index greater than 60 meets current ECHA requirements for microplastics biodegradability (2019).

### Example 6—Hair Conditioner

Selected microcapsules from the above examples are formulated into a leave-on-conditioner formulation as follows: to 98.0 grams of leave-on-conditioner (with a typical formulation given below) is added an appropriate amount of microcapsule slurry of the above examples, to deliver an encapsulated oil usage level of 0.5 wt. %. The microcapsules are added on top of the conditioner formulation, then the contents are mixed at 1000 RPM for 1 minute.

A typical composition of a leave-on conditioner formulation is given in Table 6.1 below.

TABLE 6.1
Hair Condition Formulation

| Components | Ex. I (LOT) (%) |
|---|---|
| Premix | |
| Aminosilicone | — |
| PDMS | 1.0-1.5 |
| Gel matrix carrier | |
| Behenyl trimethyl ammonium chloride | — |
| Stearamidopropyldimethylamine | 0.60-0.8 |
| (SAPDMA), C18 | |
| DTDMAC, C18(Quaternium-18) | 0.45-0.6 |
| Citric Acid (anhydrous) | 0.10-0.25 |
| Cetyl alcohol | 0.80-1.0 |
| Stearyl alcohol | 0.54-1.0 |
| Deionized Water | Balance |
| Polymers | |
| Hydroxyethylcellulose (HEC) | 0.15-0.50 |
| PEG-2M (Polyox WAR N-10) | 0.30-0.60 |
| Others | |
| Preservatives | 0.40-0.60 |

### Example 7—Shampoo

Selected microcapsules from the above examples are formulated into a rinse-off shampoo formulation as follows: to 90.0 grams of shampoo formulation is added an appropriate amount of microcapsule slurry of Examples 2 to 4, to deliver an encapsulated oil usage level of 0.5 wt. % o. The microcapsules and water are added on top of the shampoo formulation, then the contents are mixed at 1850 RPM for 1 minute. Typical shampoo formulations are shown in Tables 7.1, 7.2 and 7.3 below.

TABLE 7.1
Shampoo Formulations of Examples 7A-7C.

| | Example | | |
|---|---|---|---|
| Ingredient | 7A | 7B | 7C |
| Water | q.s. | q.s. | q.s. |
| Polyquaternium 76 ¹ | 2.50 | — | — |
| Guar, Hydroxylpropyl | — | 0.25 | — |
| Trimonium Chloride ² | | | |
| Polyquaterium 6 ³ | — | — | 0.79 |
| Sodium Laureth Sulfate (SLE3S) ⁴ | 21.43 | 21.43 | 21.43 |
| Sodium Lauryl Sulfate (SLS) ⁵ | 20.69 | 20.69 | 20.69 |
| Silicone ⁶ | 0.75 | 1.00 | 0.5 |
| Cocoamidopropyl Betaine ⁷ | 3.33 | 3.33 | 3.33 |
| Cocoamide MEA ⁸ | 1.0 | 1.0 | 1.0 |
| Ethylene Glycol Distearate ⁹ | 1.50 | 1.50 | 1.50 |
| Sodium Chloride ¹⁰ | 0.25 | 0.25 | 0.25 |
| Fragrance | 0.70 | 0.70 | 0.70 |
| Fragrance Microcapsules | 1.2 | 1.2 | 1.2 |
| Preservatives, pH adjusters | Up to 1% | Up to 1% | Up to 1% |

¹ Mirapol AT-1, Copolymer of Acrylamide(AM) and TRIQUAT, MW = 1,000,000; CD = 1.6 meq./gram; 10% active; Supplier Rhodia
² Jaguar C500, MW - 500,000, CD = 0.7, supplier Rhodia
³ Mirapol 100S, 31.5% active, supplier Rhodia
⁴ Sodium Laureth Sulfate, 28% active, supplier: P&G
⁵ Sodium Lauryl Sulfate, 29% active supplier: P&G
⁶ Glycidol Silicone VC2231-193C
⁷ Tegobetaine F-B, 30% active supplier: Goldschmidt Chemicals
⁸ Monamid CMA, 85% active, supplier Goldschmidt Chemical
⁹ Ethylene Glycol Distearate, EGDS Pure, supplier Goldschmidt Chemical
¹⁰ Sodium Chloride USP (food grade), supplier Morton; note that salt is an adjustable ingredient, higher or lower levels may be added to achieve target viscosity.

TABLE 7.2
Shampoo Formulations of Examples 7D-7F.

| | Example | | |
|---|---|---|---|
| Ingredient | 7D | 7E | 7F |
| Water | q.s. | q.s. | q.s. |
| Silicone A ¹ | 1.0 | 0.5 | 0.5 |
| Cyclopentasiloxane ⁴ | — | 0.61 | 1.5 |
| Behenyl trimethyl | 2.25 | 2.25 | 2.25 |
| ammonium chloride ⁵ | | | |
| Isopropyl alcohol | 0.60 | 0.60 | 0.60 |
| Cetyl alcohol ⁶ | 1.86 | 1.86 | 1.86 |
| Stearyl alcohol ⁷ | 4.64 | 4.64 | 4.64 |
| Disodium EDTA | 0.13 | 0.13 | 0.13 |
| NaOH | 0.01 | 0.01 | 0.01 |
| Benzyl alcohol | 0.40 | 0.40 | 0.40 |
| Methylchloroisothiazolinone/ | 0.0005 | 0.0005 | 0.0005 |
| Methylisothiazolinone ⁸ | | | |
| Panthenol ⁹ | 0.10 | 0.10 | 0.10 |
| Panthenyl ethyl ether ¹⁰ | 0.05 | 0.05 | 0.05 |
| Fragrance | 0.35 | 0.35 | 0.35 |
| Fragrance Microcapsules | 1.2 | 1.2 | 1.2 |

¹ Glycidol Silicone
⁴ Cyclopentasiloxane: SF1202 available from Momentive Performance Chemicals
⁵ Behenyl trimethyl ammonium chloride/Isopropyl alcohol: Genamin TM KMP available from Clariant
⁶ Cetyl alcohol: Konol TM series available from Shin Nihon Rika
⁷ Stearyl alcohol: Konol TM series available from Shin Nihon Rika
⁸ Methylchloroisothiazolinone/Methylisothiazolinone: Kathon TM CG available from Rohm & Haas
⁹ Panthenol: Available from Roche
¹⁰ Panthenyl ethyl ether: Available from Roche

TABLE 7.3
Shampoo Formulations of Examples 7G and 7H

| | Example | |
|---|---|---|
| Ingredient | 7G | 7H |
| Sodium Laureth Sulfate | 10.00 | 10.00 |
| Sodium Lauryl Sulfate | 1.50 | 1.50 |
| Cocamidopropyl betaine | 2.00 | 2.00 |
| Guar Hydroxypropyl trimonium chloride ¹ | 0.40 | |
| Guar Hydroxypropyl trimonium chloride ² | | 0.40 |
| Dimethicone ³ | 2.00 | 2.00 |
| Gel Network ⁴ | | 27.27 |
| Ethylene Glycol Distearate | 1.50 | 1.50 |
| 5-Chloro-2-methyl-4- | 0.0005 | 0.0005 |
| isothiazolin-3-one, Kathon CG | | |
| Sodium Benzoate | 0.25 | 0.25 |
| Disodium EDTA | 0.13 | 0.13 |
| Perfume | 0.40 | 0.40 |
| Fragrance Microcapsules | 0.30 | 0.30 |
| Citric Acid/Sodium Citrate Dihydrate | pH QS | pH QS |
| Sodium Chloride/Ammonium Xylene Sulfonate | Visc. QS | Visc. QS |
| Water | QS | QS |

¹ Jaguar C17 available from Rhodia
² N-Hance 3269 (with Mol. W. of ~500,000 and 0.8 meq/g) available from Aqulaon/Hercules
³ Viscasil 330M available from General Electric Silicones
⁴ Gel Networks; See composition in Table 7.4 below. The water is heated to about 74° C. and the Cetyl Alcohol, Stearyl Alcohol, and the SLES Surfactant are added to it. After incorporation, this mixture is passed through a heat exchanger where it is cooled to about 35° C. As a result of this cooling step, the Fatty Alcohols and surfactant crystallized to form a crystalline gel network.

TABLE 7.4
Gel Network Composition

| Ingredient | Wt. % |
|---|---|
| Water | 86.14% |
| Cetyl Alcohol | 3.46% |
| Stearyl Alcohol | 6.44% |
| Sodium laureth-3 sulfate (28% Active) | 3.93% |
| 5-Chloro-2-methyl-4- | 0.03% |
| isothiazolin-3-one, Kathon CG | |

### Example 8—Lotion

For the examples shown in Table 8 below, in a suitable container, combine the ingredients of Phase A. In a separate suitable container, combine the ingredients of Phase B. Heat each phase to 73C-78° C. while mixing each phase using a suitable mixer (e.g., Anchor blade, propeller blade, or IKA T25) until each reaches a substantially constant desired temperature and is homogenous. Slowly add Phase B to Phase A while continuing to mix Phase A. Continue mixing until batch is uniform. Pour product into suitable containers at 73-78° C. and store at room temperature. Alternatively, continuing to stir the mixture as temperature decreases results in lower observed hardness values at 21 and 33° C.

TABLE 8
Lotion Formulations (Examples 8A-8C).

| | Example | | |
|---|---|---|---|
| Ingredient/Property | 8A | 8B | 8C |
| PHASE A | | | |
| DC-9040 ¹ | 8.60 | 3.00 | 5.00 |
| Dimethicone | 4.09 | 4.00 | 4.00 |
| Polymethylsilsesquioxane ² | 4.09 | 4.00 | 4.00 |
| Cyclomethicone | 11.43 | 0.50 | 11.33 |
| KSG-210 ³ | 5.37 | 5.25 | 5.40 |
| Polyethylene wax ⁴ | 3.54 | | 2.05 |
| DC-2503 Cosmetic Wax ⁵ | 7.08 | 10.00 | 3.77 |
| Hydrophobic TiO₂ | | | 0.50 |
| Iron oxide coated Mica | | | 0.65 |
| TiO₂ Coated Mica | 1.00 | 1.00 | |
| Fragrance Microcapsules | 1.00 | 1.00 | 1.00 |
| PHASE B | | | |
| Glycerin | 10.00 | 10.00 | 10.00 |
| Dexpanthenol | 0.50 | 0.50 | 0.50 |
| Pentylene Glycol | 3.00 | 3.00 | 3.00 |
| Hexamidine Diisethionate ⁶ | 0.10 | 0.10 | 0.10 |
| Niacinamide ⁷ | 5.00 | 5.00 | 5.00 |
| Methylparaben | 0.20 | 0.20 | 0.20 |
| Ethylparaben | 0.05 | 0.05 | 0.05 |
| Sodium Citrate | 0.20 | 0.20 | 0.20 |
| Citric Acid | 0.03 | 0.03 | 0.03 |
| Sodium Benzoate | 0.05 | 0.05 | 0.05 |
| Sodium Chloride | 0.50 | 0.50 | 0.50 |
| FD&C Red #40 (1%) | 0.05 | 0.05 | 0.05 |
| Water | q.s to 100 | q.s to 100 | q.s to 100 |
| Hardness at 21° C. (g) | 33.3 | 15.4 | 14.2 |
| Hardness at 33° C. (g) | 6.4 | 0.7 | 4.0 |

¹ 12.5% Dimethicone Crosspolymer in Cyclopentasiloxane. Available from Dow Corning.
² E.g., TOSPEAR 145A or TOSPEARL 2000. Available from GE Toshiba Silicon.
³ 25% Dimethicone PEG-10/15 Crosspolymer in Dimethicone. Available from Shin-Etsu.
⁴ JEENATE 3H polyethylene wax from Jeen.
⁵ Stearyl Dimethicone. Available from Dow Corning.
⁶ Hexamidine diisethionate, available from Laboratoires Serobiologiques.
⁷ Additionally or alternatively, the composition may comprise one or more other skin care actives, their salts and derivatives, as disclosed herein, in amounts also disclosed herein as would be deemed suitable by one of skill in the art.

### Example 9—Antiperspirant/Deodorant

Example 9A of Table 9.1 below can be made via the following general process, which one skilled in the art will be able to alter to incorporate available equipment. The ingredients of Part I and Part II are mixed in separate suitable containers. Part II is then added slowly to Part I under agitation to assure the making of a water-in-silicone emulsion. The emulsion is then milled with a suitable mill, for example a Greeco 1L03 from Greeco Corp, to create a homogenous emulsion. Part III is mixed and heated to 88° C. until the all solids are completely melted. The emulsion is then also heated to 88° C. and then added to the Part 3 ingredients. The final mixture is then poured into an appropriate container, and allowed to solidify and cool to ambient temperature.

TABLE 9.1
Antiperspirant/Deodorant Formulation (Example 9A).

| Ingredient | Example 9A |
|---|---|
| Part I: Partial Continuous Phase | |
| Hexamethyldisiloxane¹ | QS |
| DC5200² | 1.20 |
| Fragrance | 0.35 |
| Fragrance Capsules | 1.00 |
| Part II: Disperse Phase | |
| ACH (40% solution)³ | 40.00 |
| propylene glycol | 5.00 |
| Water | 12.30 |
| Part III: Structurant Plus Remainder | |
| of Continuous Phase | |
| FINSOLVE TN | 6.50 |

QS—indicates that this material is used to bring the total to 100%.
¹DC 246 fluid from Dow Corning
²from Dow Corning
³Standard aluminum chlorohydrate solution

Examples 9B to 9E of Table 9.2 below can be made as follows: all ingredients except the fragrance, and fragrance capsules are combined in a suitable container and heated to about 85C to form a homogenous liquid. The solution is then cooled to about 62C and then the fragrance, and fragrance microcapsules are added. The mixture is then poured into an appropriate container and allowed to solidify up cooling to ambient temperature.

Example 9F of Table 9.2 can be made as follows: all the ingredients except the propellant are combined in an appropriate aerosol container. The container is then sealed with an appropriate aerosol delivery valve. Next air in the container is removed by applying a vacuum to the valve and then propellant is added to container through the valve. Finally an appropriate actuator is connected to the valve to allow dispensing of the product.

TABLE 9.2
Antiperspirant/Deodorant Formulations

| | Example | | | | |
|---|---|---|---|---|---|
| Ingredient | 9B | 9C | 9D | 9E | 9F |
| Product Form | Solid | Solid | Solid | Solid | Deodorant |
| | Deodorant | Deodorant | Deodorant | Deodorant | or Body |
| | | | | | Spray |
| dipropylene glycol | 45 | 22 | 20 | 30 | 20 |
| propylene glycol | 22 | 45 | 22 | | |
| tripopylene glycol | | | 25 | | |
| Glycerine | | | | 10 | |
| PEG-8 | | | | 20 | |
| ethanol | | | | | QS |
| Water | QS | QS | QS | QS | |
| sodium stearate | 5.5 | 5.5 | 5.5 | 5.5 | |
| tetra sodium EDTA | 0.05 | 0.05 | 0.05 | 0.05 | |
| sodium hydroxide | 0.04 | 0.04 | 0.04 | 0.04 | |
| triclosan | 0.3 | 0.3 | 0.3 | 0.3 | |
| Fragrance | 0.5 | 0.5 | 0.5 | 0.5 | 0.5 |
| Fragrance capsules | 1.0 | 1.0 | 1.0 | 1.0 | 0.5 |
| Propellant (1,1 | | | | | 40 |
| difluoroethane) | | | | | |

QS—indicates that this material is used to bring the total to 100%.

### Example 10—Rinse-off Conditioner

The conditioning compositions of Examples 10A through 10F Table 10 are prepared as follows: cationic surfactants, high melting point fatty compounds are added to water with agitation, and heated to about 80° C. The mixture is cooled down to about 50° C. to form a gel matrix carrier. Separately, slurries of perfume microcapsules and silicones are mixed with agitation at room temperature to form a premix. The premix is added to the gel matrix carrier with agitation. If included, other ingredients such as preservatives are added with agitation. Then the compositions are cooled down to room temperature.

The conditioning composition of Example 10B of Table 10 is prepared as follows: cationic surfactants, high melting point fatty compounds are added to water with agitation, and heated to about 80° C. The mixture is cooled down to about 50° C. to form a gel matrix carrier. Then, silicones are added with agitation. Separately, slurries of perfume microcapsules, and if included, other ingredients such as preservatives are added with agitation. Then the compositions are cooled down to room temperature.

TABLE 10
Rinse-Off Conditioner Formulations (Examples 10A-10F).

| | Example | | | | | |
|---|---|---|---|---|---|---|
| Ingredient | 10A | 10B | 10C | 10D | 10E | 10F³ |
| Premix | | | | | | |
| Aminosilicone-1¹ | 0.50 | 0.50 | | | | |
| Aminosilicone-2 ² | | | 0.50 | 0.50 | 0.50 | |
| PDMS | | | | | | 0.50 |
| Fragrance microcapsules | . . . | 1.0 | 1.0 | 1.0 | 1.0 | 1.0 |
| Gel matrix carrier | | | | | | |
| Behenyl trimethyl ammonium | 2.30 | 2.30 | 2.30 | 2.30 | 2.30 | 2.30 |
| chloride | | | | | | |
| Cetyl alcohol | 1.5 | 1.5 | 1.5 | 1.5 | 1.5 | 1.5 |
| Stearyl alcohol | 3.8 | 3.8 | 3.8 | 3.8 | 3.8 | 3.8 |
| Deionized Water | QS | QS | QS | QS | QS | QS |
| Preservatives | 0.4 | 0.4 | 0.4 | 0.4 | 0.4 | 0.4 |
| Panthenol | — | — | 0.03 | — | — | — |
| Panthenyl ethyl ether | — | — | 0.03 | — | — | — |

¹Aminosilicone-1 (AMD): having an amine content of 0.12-0.15 m mol/g and a viscosity of 3,000-8,000 mPa · s, which is water insoluble
² Aminosilicone-2 (TAS): having an amine content of 0.04-0.06 m mol/g and a viscosity of 10,000-16,000 mPa · s, which is water insoluble
³Comparative example with PDMS instead of amino silicone

### Example 11—Body Cleansing Composition

The body cleaning compositions of Examples 11A-11C are prepared as follows.

The cleansing phase composition is prepared by adding surfactants, guars, and Stabylen 30 to water. Sodium chloride is then added to the mixture to thicken the cleansing phase composition. Preservatives and chelants are added to the formulation. Finally, perfume is added to the suspension.

The benefit phase composition is prepared by mixing petrolatum and mineral oil to make a homogeneous mixture. Fragrance microcapsules are added to the suspension. Finally, the cleansing phase (e.g. surfactant phase) and benefit phase are mixed in different ratios to yield the body cleansing composition.

TABLE 11
Body Cleansing Composition Formulations (Examples 11A-11C).

| | Example | | |
|---|---|---|---|
| Ingredient | 11A | 11B | 11C |
| I: Cleansing Phase Composition | | | |
| Sodium Trideceth Sulfate | 5.9 | 5.9 | 5.9 |
| (sulfated from Iconol TDA-3 (BASF | | | |
| Corp.) to >95% sulfate) | | | |
| Sodium Lauryl Sulfate | 5.9 | 5.9 | 5.9 |
| Sodium Lauroamphoacetate | 3.6 | 3.6 | 3.6 |
| (Cognis Chemical Corp.,) | | | |
| Guar Hydroxypropyltrimonium | — | 0.3 | 0.7 |
| Chloride | | | |
| (N-Hance 3196 from Aqualon) | | | |
| Guar Hydroxypropyltrimonium | 0.6 | — | — |
| Chloride | | | |
| (Jaguar C-17 from Rhodia) | | | |
| Stabylen 30 | 0.33 | 0.33 | 0.33 |
| (Acrylates/Vinyl Isodecanoate, 3V) | | | |
| Sodium Chloride | 3.75 | 3.75 | 3.75 |
| Trideceth-3 | 1.75 | 1.75 | 1.75 |
| (Iconal TDA-3 from BASF Corp.) | | | |
| Methyl chloro isothiazolinone and | 0.033 | 0.033 | 0.033 |
| methyl isothiazolinone (Kathon CG, | | | |
| Rohm & Haas) | | | |
| EDTA (Dissolvine NA 2x) | 0.15 | 0.15 | 0.15 |
| Sodium Benzoate | 0.2 | 0.2 | 0.2 |
| Citric Acid, titrate | pH = | pH = | pH = |
| | 5.7 ± 0.2 | 5.7 ± 0.2 | 5.7 ± 0.2 |
| Perfume | 1.11% | 1.11% | 1.11% |
| Water and Minors (NaOH) | Q.S. | Q.S. | Q.S. |
| II: Benefit Phase Composition | | | |
| Petrolatum | 60 | 60 | 60 |
| (G2218 from Sonnerbonn) | | | |
| Mineral Oil | 20 | 20 | 20 |
| (Hydrobrite 1000 from Sonnerbonn) | | | |
| Fragrance Microcapsules | 10 | 10 | 10 |
| III: Surfactant Phase:Benefit | 50:50 | 90:10 | 90:10 |
| Phase Blending Ratio | | | |

### Example 12—Fabric Softening Product

Non-limiting examples of product formulations containing purified perfume microcapsules of the aforementioned examples are summarized in the following table.

TABLE 12
Fabric Softening Product Formulations (Examples 12A-12J).

| | Example | | | | | | | | | |
|---|---|---|---|---|---|---|---|---|---|---|
| Ingredient | 12A | 12B | 12C | 12D | 12E | 12F | 12G | 12H | 12I | 12J |
| FSA ^{a} | 14 | 16.47 | 14 | 12 | 12 | 16.47 | 3.00 | 6.5 | 5 | 5 |
| Ethanol | 2.18 | 2.57 | 2.18 | 1.95 | 1.95 | 2.57 | — | — | 0.81 | 0.81 |
| Isopropyl | — | — | — | — | — | — | 0.33 | 1.22 | — | — |
| Alcohol | | | | | | | | | | |
| Microcapsule | 0.6 | 0.75 | 0.6 | 0.75 | 0.37 | 0.60 | 0.37 | 0.6 | 0.37 | 0.37 |
| (% active)* | | | | | | | | | | |
| Phase | 0.21 | 0.25 | 0.21 | 0.21 | 0.14 | — | — | 0.14 | — | — |
| Stabilizing | | | | | | | | | | |
| Polymer ^{f} | | | | | | | | | | |
| Suds Suppressor ^{g} | — | — | — | — | — | — | — | 0.1 | — | — |
| Calcium | 0.15 | 0.176 | 0.15 | 0.15 | 0.30 | 0.176 | — | 0.1-0.15 | — | — |
| Chloride | | | | | | | | | | |
| DTPA ^{h} | 0.017 | 0.017 | 0.017 | 0.017 | 0.007 | 0.007 | 0.20 | — | 0.002 | 0.002 |
| Preservative | 5 | 5 | 5 | 5 | 5 | 5 | — | 250 ^{j} | 5 | 5 |
| (ppm) ^{i, j} | | | | | | | | | | |
| Antifoam^{k} | 0.015 | 0.018 | 0.015 | 0.015 | 0.015 | 0.015 | — | — | 0.015 | 0.015 |
| Dye | 40 | 40 | 40 | 40 | 40 | 40 | 11 | 30-300 | 30 | 30 |
| (ppm) | | | | | | | | | | |
| Ammonium | 0.100 | 0.118 | 0.100 | 0.100 | 0.115 | 0.115 | — | — | — | — |
| Chloride | | | | | | | | | | |
| HCl | 0.012 | 0.014 | 0.012 | 0.012 | 0.028 | 0.028 | 0.016 | 0.025 | 0.011 | 0.011 |
| Structurant^{l} | 0.01 | 0.01 | 0.01 | 0.01 | 0.01 | 10.01 | 0.01 | 0.01 | 0.01 | 0.01 |
| Neat | 0.8 | 0.7 | 0.9 | 0.5 | 1.2 | 0.5 | 1.1 | 0.6 | 1.0 | 0.9 |
| Unencapsulated | | | | | | | | | | |
| Perfume | | | | | | | | | | |
| Deionized | Balance | Balance | Balance | Balance | Balance | Balance | Balance | Balance | Balance | Balance |
| Water | | | | | | | | | | |

^{a} N,N-di(tallowoyloxyethyl)-N,N-dimethylammonium chloride.
^{f} Copolymer of ethylene oxide and terephthalate having the formula described in U.S. Pat. No. 5,574,179 at col. 15, lines 1-5, wherein each X is methyl, each n is 40, u is 4, each R1 is essentially 1,4-phenylene moieties, each R2 is essentially ethylene, 1,2-propylene moieties, or mixtures thereof.
^{g} SE39 from Wacker
^{h} Diethylenetriaminepentaacetic acid.
ⁱ KATHON CG available from Rohm and Haas Co. “PPM” is “parts per million.”
^{j} Gluteraldehyde
^{k}Silicone antifoam agent available from Dow Corning Corp. under the trade name DC2310.
^{l}Hydrophobically-modified ethoxylated urethane available from Rohm and Haas under the tradename Aculyn ™ 44.
*Suitable microcapsules provided in Examples 2 to 4. (Percent active relates to the core content of the microcapsule)

### Example 13—Dry Laundry Formulations

Non-limiting examples of product formulations containing purified perfume microcapsules of the aforementioned examples are summarized in the following table.

TABLE 13
Dry Laundry Formulations (Examples 13A-13G)

| | % w/w granular laundry detergent composition | | | | | | |
|---|---|---|---|---|---|---|---|
| | Example | | | | | | |
| Ingredient | 13A | 13B | 13C | 13D | 13E | 13F | 13G |
| Brightener | 0.1 | 0.1 | 0.1 | 0.2 | 0.1 | 0.2 | 0.1 |
| Soap | 0.6 | 0.6 | 0.6 | 0.6 | 0.6 | 0.6 | 0.6 |
| Ethylenediamine disuccinic acid | 0.1 | 0.1 | 0.1 | 0.1 | 0.1 | 0.1 | 0.1 |
| Acrylate/maleate copolymer | 1.5 | 1.5 | 1.5 | 1.5 | 1.5 | 1.5 | 1.5 |
| Hydroxyethane di(methylene | 0.4 | 0.4 | 0.4 | 0.4 | 0.4 | 0.4 | 0.4 |
| phosphonic acid) | | | | | | | |
| Mono-C₁₂₋₁₄ alkyl, di-methyl, | 0.5 | 0.5 | 0.5 | 0.5 | 0.5 | 0.5 | 0.5 |
| mono-hydroyethyl quaternary | | | | | | | |
| ammonium chloride | | | | | | | |
| Linear alkyl benzene | 0.1 | 0.1 | 0.2 | 0.1 | 0.1 | 0.2 | 0.1 |
| Linear alkyl benzene sulphonate | 10.3 | 10.1 | 19.9 | 14.7 | 10.3 | 17 | 10.5 |
| Magnesium sulphate | 0.4 | 0.4 | 0.4 | 0.4 | 0.4 | 0.4 | 0.4 |
| Sodium carbonate | 19.5 | 19.2 | 10.1 | 18.5 | 29.9 | 10.1 | 16.8 |
| Sodium sulphate | QS | QS | QS | QS | QS | QS | QS |
| Sodium Chloride | 0.1 | 0.1 | 0.1 | 0.1 | 0.1 | 0.1 | 0.1 |
| Zeolite | 9.6 | 9.4 | 8.1 | 18 | 10 | 13.2 | 17.3 |
| Photobleach particle | 0.1 | 0.1 | 0.2 | 0.1 | 0.2 | 0.1 | 0.2 |
| Blue and red carbonate speckles | 1.8 | 1.8 | 1.8 | 1.8 | 1.8 | 1.8 | 1.8 |
| Ethoxylated Alcohol AE7 | 1 | 1 | 1 | 1 | 1 | 1 | 1 |
| Tetraacetyl ethylene diamine | 0.9 | 0.9 | 0.9 | 0.9 | 0.9 | 0.9 | 0.9 |
| agglomerate (92 wt. % active) | | | | | | | |
| Citric acid | 1.4 | 1.4 | 1.4 | 1.4 | 1.4 | 1.4 | 1.4 |
| Polyethylene oxide | 0.2 | 0.2 | 0.2 | 0.2 | 0.2 | 0.2 | 0.2 |
| Enzymes e.g. Protease (84 mg/g | 0.2 | 0.3 | 0.2 | 0.1 | 0.2 | 0.1 | 0.2 |
| active), Amylase (22 mg/g active) | | | | | | | |
| Suds suppressor agglomerate | 0.2 | 0.2 | 0.2 | 0.2 | 0.2 | 0.2 | 0.2 |
| (12.4 wt. % active) | | | | | | | |
| Sodium percarbonate (having | 7.2 | 7.1 | 4.9 | 5.4 | 6.9 | 19.3 | 13.1 |
| from 12% to 15% active AvOx) | | | | | | | |
| Perfume oil | 0.5 | 0.5 | 0.5 | 0.5 | 0.5 | 0.5 | 0.5 |
| Solid perfume particles | 0.4 | 0 | 0.4 | 0.4 | 0.4 | 0.4 | 0.6 |
| Perfume microcapsules | 1.3 | 2.4 | 1 | 1.3 | 1.3 | 1.3 | 0.7 |
| (Example 2 to 4) | | | | | | | |
| Water | 1.4 | 1.4 | 1.4 | 1.4 | 1.4 | 1.4 | 1.4 |
| Misc | 0.1 | 0.1 | 0.1 | 0.1 | 0.1 | 0.1 | 0.1 |
| Total Parts | 100 | 100 | 100 | 100 | 100 | 100 | 100 |

QS—as used herein indicates that this material is used to bring the total to 100%.

### Example 14—Liquid Laundry Formulations (HDLs)

Non-limiting examples of product formulations containing purified perfume microcapsules of the aforementioned examples are summarized in Tables 14.1, 14.2 and 14.3 below.

TABLE 14.1
Liquid Laundry Formulations (HDLs)

| | Example | | | | | |
|---|---|---|---|---|---|---|
| Ingredient | 14A | 14B | 14C | 14D | 14E | 14F |
| Alkyl Ether Sulphate | 0.00 | 0.50 | 12.0 | 12.0 | 6.0 | 7.0 |
| Dodecyl Benzene | 8.0 | 8.0 | 1.0 | 1.0 | 2.0 | 3.0 |
| Sulphonic Acid | | | | | | |
| Ethoxylated Alcohol | 8.0 | 6.0 | 5.0 | 7.0 | 5.0 | 3.0 |
| Citric Acid | 5.0 | 3.0 | 3.0 | 5.0 | 2.0 | 3.0 |
| Fatty Acid | 3.0 | 5.0 | 5.0 | 3.0 | 6.0 | 5.0 |
| Ethoxysulfated | 1.9 | 1.2 | 1.5 | 2.0 | 1.0 | 1.0 |
| hexamethylene diamine | | | | | | |
| quaternized | | | | | | |
| Diethylene triamine penta | 0.3 | 0.2 | 0.2 | 0.3 | 0.1 | 0.2 |
| methylene phosphonic | | | | | | |
| acid | | | | | | |
| Enzymes | 1.20 | 0.80 | 0 | 1.2 | 0 | 0.8 |
| Brightener (disulphonated | 0.14 | 0.09 | 0 | 0.14 | 0.01 | 0.09 |
| diamino stilbene based | | | | | | |
| FWA) | | | | | | |
| Cationic hydroxyethyl | 0 | 0 | 0.10 | 0 | 0.200 | 0.30 |
| cellulose | | | | | | |
| Poly(acrylamide-co- | 0 | 0 | 0 | 0.50 | 0.10 | 0 |
| diallyldimethylammonium | | | | | | |
| chloride) | | | | | | |
| Hydrogenated Castor Oil | 0.50 | 0.44 | 0.2 | 0.2 | 0.3 | 0.3 |
| Structurant | | | | | | |
| Boric acid | 2.4 | 1.5 | 1.0 | 2.4 | 1.0 | 1.5 |
| Ethanol | 0.50 | 1.0 | 2.0 | 2.0 | 1.0 | 1.0 |
| 1,2 propanediol | 2.0 | 3.0 | 1.0 | 1.0 | 0.01 | 0.01 |
| Diethyleneglycol (DEG) | 1.6 | 0 | 0 | 0 | 0 | 0 |
| 2,3-Methyl-1,3- | 1.0 | 1.0 | 0 | 0 | 0 | 0 |
| propanediol (M pdiol) | | | | | | |
| Mono Ethanol Amine | 1.0 | 0.5 | 0 | 0 | 0 | 0 |
| NaOH Sufficient To | pH 8 | pH 8 | pH 8 | pH 8 | pH 8 | pH 8 |
| Provide Formulation pH | | | | | | |
| of: | | | | | | |
| Sodium Cumene | 2.00 | 0 | 0 | 0 | 0 | 0 |
| Sulphonate (NaCS) | | | | | | |
| Perfume | 0.7 | 0.5 | 0.8 | 0.8 | 0.6 | 0.6 |
| Polyethyleneimine | 0.01 | 0.10 | 0.00 | 0.10 | 0.20 | 0.05 |
| Perfume Microcapsules | 1.00 | 5.00 | 1.00 | 2.00 | 0.10 | 0.80 |
| of Example 2 to 4 | | | | | | |
| Water | Balance | Balance | Balance | Balance | Balance | Balance |
| | to 100% | to 100% | to 100% | to 100% | to 100% | to 100% |

TABLE 14.2
Liquid Laundry Detergent Formulations

| | Example | | | |
|---|---|---|---|---|
| Ingredient | 14G | 14H | 14I | 14J |
| C14-C15 alkyl poly | 6.25 | 4.00 | 6.25 | 6.25 |
| ethoxylate (8) | | | | |
| C12-C14 alkyl poly | 0.40 | 0.30 | 0.40 | 0.40 |
| ethoxylate (7) | | | | |
| C12-C14 alkyl poly | 10.60 | 6.78 | 10.60 | 10.60 |
| ethoxylate (3) | | | | |
| sulfate Na salt | | | | |
| Linear Alkylbenzene | 0.19 | 1.16 | 0.79 | 0.79 |
| sulfonate acid | | | | |
| Citric Acid | 3.75 | 2.40 | 3.75 | 3.75 |
| C12-C18 Fatty Acid | 4.00 | 2.56 | 7.02 | 7.02 |
| Enzymes | 0.60 | 0.4 | 0.60 | 0.60 |
| Boric Acid | 2.4 | 1.5 | 1.25 | 1.25 |
| Trans-sulphated | 1.11 | 0.71 | 1.11 | 1.11 |
| ethoxylated | | | | |
| hexamethylene | | | | |
| diamine quat | | | | |
| Diethylene triamine | 0.17 | 0.11 | 0.17 | 0.17 |
| penta methylene | | | | |
| phosphonic acid | | | | |
| Fluorescent brightener | 0.09 | 0.06 | 0.14 | 0.14 |
| Hydrogenated Castor Oil | 0.05 | 0.300 | 0.20 | 0.20 |
| Ethanol | 2.50 | 1.00 | 2.50 | 2.50 |
| 1,2 propanediol | 1.14 | 0.7 | 1.14 | 1.14 |
| Sodium hydroxide | 3.8 | 2.6 | 4.60 | 4.60 |
| Mono Ethanol Amine | 0.8 | 0.5 | | |
| Na Cumene Sulphonate | | | 1.0 | |
| Dye | 0.002 | 0.002 | 0.002 | 0.002 |
| Opacifier (Styrene | 0.1 | | | |
| Acrylate based) | | | | |
| Bentonite Softening Clay | | 1.0 | | |
| Polyquaternium 10 - | 1.0 | | 1.0 | 1.0 |
| Cationic hydroxyl | | | | |
| ethyl cellulose | | | | |
| PP-5495 (silicone | | 1.0 | | |
| ex Dow Corning | | | | |
| Corporation, | | | | |
| Midland, MI) | | | | |
| DC 1664 (silicone | | | 1.0 | |
| ex Dow Corning | | | | |
| Corporation, | | | | |
| Midland, MI) | | | | |
| Perfume micro capsules | 0.8 | 0.5 | 1.0 | 0.7 |
| (expressed as perfume | | | | |
| oil) of Example 2 to 4 | | | | |
| Perfume | 0.7 | 0.55 | 1.00 | 1.00 |
| Poly Ethylene | 0.1 | | | |
| Imine MW 25000 | | | | |
| Water | Up to 100 | Up to 100 | Up to 100 | Up to 100 |

TABLE 14.3
Liquid Laundry Detergent Formulations.

| | Example | | |
|---|---|---|---|
| Ingredient | 14K | 14L | 14M |
| C14-C15 alkyl poly | 3.7 | | 20.7 |
| ethoxylate (8) | | | |
| C12-C14 alkyl poly | | 16.7 | |
| ethoxylate (7) | | | |
| C12-C14 alkyl poly | 17.8 | | 5.5 |
| ethoxylate (3) | | | |
| sulfate Na salt | | | |
| Linear Alkylbenzene | 12.5 | 22.9 | 13.5 |
| sulfonate acid | | | |
| Citric Acid | 3.9 | | 1.7 |
| C12-C18 Fatty Acid | 11.1 | 18 | 5.1 |
| Enzymes | 3 | 1.2 | 3 |
| Boric Acid | 0.5 | | 0.5 |
| Trans-sulphated ethoxylated | 3.25 | | 1.2 |
| hexamethylene | | | |
| diamine quat | | | |
| PEI 600 EO20 | 1.25 | | 1.2 |
| Diethylene triamine penta | 1.6 | | 0.85 |
| methylene phosphonic | | | |
| acid or HEDP | | | |
| Fluorescent brightener | 0.2 | 0.3 | 0.14 |
| Hydrogenated Castor Oil | | 0.2 | |
| 1,2 propanediol | 4.3 | 20.3 | 11.7 |
| Sodium hydroxide | | 1.0 | 3.9 |
| Mono Ethanol Amine | 9.8 | 6.8 | 3.1 |
| Dye | Present | Present | Present |
| PDMS | | 2.15 | |
| Potassium sulphite | | 0.2 | |
| Perfume micro capsules | 1.6 | 1.5 | 1.4 |
| (expressed as perfume | | | |
| oil) of Examples 2 to 4 | | | |
| Perfume | 1.2 | 1.6 | 1.0 |
| Form. Phenyl Boronic Acid | | | Present |
| Water** | Up to 100 | Up to 100 | Up to 100 |

**Low water liquid detergent in Polyvinylalcohol unidose/sachet

### Example 15—Liquid and Gel Detergents

Non-limiting examples of product formulations containing purified perfume microcapsules of the aforementioned examples are summarized in Table 15 below.

TABLE 15
Liquid and Gel Detergent Formulations (% by Weight)

| | Example | | |
|---|---|---|---|
| Ingredient | 15A | 15B | 15C |
| Alkylbenzenesulfonic acid | 17.2 | 12.2 | 23 |
| C12-14 alcohol 7-ethoxylate | 8.6 | 0.4 | 19.5 |
| C14-15 alcohol 8-ethoxylate | — | 9.6 | — |
| C12-14 alcohol 3-ethoxylate | 8.6 | — | — |
| sulphate, Na salt | | | |
| C8-10 Alkylamidopropyldimethyl amine | — | — | 0.9 |
| Citric acid | 2.9 | 4.0 | — |
| C12-18 fatty acid | 12.7 | 4.0 | 17.3 |
| Enzymes | 3.5 | 1.1 | 1.4 |
| Ethoxylated polyimine | 1.4 | — | 1.6 |
| Ethoxylated polyimine | 3.7 | 1.8 | 1.6 |
| polymer, quaternized | | | |
| and sulphated | | | |
| Hydroxyethane diphosphonic | 1.4 | — | — |
| acids (HEDP) | | | |
| Pentamethylene triamine | — | 0.3 | — |
| pentaphosphonic acid | | | |
| Catechol 2,5 disulfonate, Na salt | 0.9 | — | — |
| Fluorescent whitening agent | 0.3 | 0.15 | 0.3 |
| 1,2 propandiol | 3.5 | 3.3 | 22 |
| Ethanol | — | 1.4 | — |
| Diethylene glycol | — | 1.6 | — |
| 1-ethoxypentanol | 0.9 | — | — |
| Sodium cumene sulfonate | | 0.5 | — |
| Monoethanolamine (MEA) | 10.2 | 0.8 | 8.0 |
| MEA borate | 0.5 | 2.4 | — |
| Sodium hydroxide | — | 4.6 | — |
| Perfume | 1.6 | 0.7 | 1.5 |
| Perfume microcapsules | 1.1 | 1.2 | 0.9 |
| as Example 2 to 4 | | | |
| Water | 22.1 | 50.8 | 2.9 |
| Perfume, dyes, miscellaneous minors | Balance | Balance | Balance |
| Undiluted viscosity | 2700 | 400 | 300 |
| (Vₙ) at 20 s⁻¹, cps | | | |

### Example 16—Liquid Unit Dose

The following are examples of unit dosage forms wherein the liquid composition is enclosed within a PVA film. The preferred film used in the present examples is Monosol M8630 76 μm thickness.

TABLE 16
Unit Dose Laundry Cleaner

| | Example | | | | | | | |
|---|---|---|---|---|---|---|---|---|
| | 16A | | | 16B | | 16C | | |
| | 3 compartments | | | 2 compartments | | 3 compartments | | |
| | Compartment # | | | | | | | |
| | 42 | 43 | 44 | 45 | 46 | 47 | 48 | 49 |
| | Dosage (g) | | | | | | | |
| | 34.0 | 3.5 | 3.5 | 30.0 | 5.0 | 25.0 | 1.5 | 4.0 |
| Ingredients | Weight % | | | | | | | |
| Alkylbenzene sulfonic acid | 20.0 | 20.0 | 20.0 | 10.0 | 20.0 | 20.0 | 25 | 30 |
| Alkyl sulfate | | | | 2.0 | | | | |
| C₁₂₋₁₄ alkyl 7-ethoxylate | 17.0 | 17.0 | 17.0 | | 17.0 | 17.0 | 15 | 10 |
| C₁₂₋₁₄ alkyl ethoxy 3 sulfate | 7.5 | 7.5 | 7.5 | | | 7.5 | 7.5 | |
| Citric acid | 0.5 | | 2.0 | 1.0 | | | | 2.0 |
| Zeolite A | | | | 10.0 | | | | |
| C₁₂₋₁₈ Fatty acid | 13.0 | 13.0 | 13.0 | | 18.0 | 18.0 | 10 | 15 |
| Sodium citrate | | | | 4.0 | 2.5 | | | |
| Enzymes | 0-3 | 0-3 | 0-3 | 0-3 | | 0-3 | 0-3 | 0-3 |
| Sodium Percarbonate | | | | 11.0 | | | | |
| TAED | | | | 4.0 | | | | |
| Polycarboxylate | | | | 1.0 | | | | |
| Ethoxylated | 2.2 | 2.2 | 2.2 | | | | | |
| Polyethylenimine¹ | | | | | | | | |
| Hydroxyethane | 0.6 | 0.6 | 0.6 | 0.5 | | | 2.2 | |
| diphosphonic acid | | | | | | | | |
| Ethylene diamine | | | | | | 0.4 | | |
| tetra(methylene phosphonic) | | | | | | | | |
| acid | | | | | | | | |
| Brightener | 0.2 | 0.2 | 0.2 | 0.3 | | 0.3 | | |
| Microcapsules of examples | 0.4 | 1.2 | 1.5 | 1.3 | 1.3 | 0.4 | 0.12 | 0.2 |
| 2 to 4 | | | | | | | | |
| Water | 9 | 8.5 | 10 | 5 | 11 | 10 | 10 | 9 |
| CaCl2 | | | | | | | 0.01 | |
| Perfume | 1.7 | 1.7 | | 0.6 | | 1.5 | 0.5 | |
| Minors (antioxidant, sulfite, | 2.0 | 2.0 | 2.0 | 4.0 | 1.5 | 2.2 | 2.2 | 2.0 |
| aesthetics, . . .) | | | | | | | | |

| | |
|---|---|
| Buffers (sodium | To pH 8.0 for liquids |
| carbonate, | To RA > 5.0 for powders |
| monoethanolamine) ² | |
| Solvents (1,2 propanediol, | To 100p |
| ethanol), sodium sulfate | |

¹Polyethylenimine (MW = 600) with 20 ethoxylate groups per —NH.
²RA = Reserve Alkalinity (g NaOH/dose)

While the invention has been described in detail and with reference to specific examples thereof, it will be apparent to one skilled in the art that various changes and modifications can be made therein without departing from the spirit and scope thereof.

What is claimed is:

1. A resin composition comprising a macromolecule crosslinked with polyamide epichlorohydrin, wherein the macromolecule is at least one member selected from the group consisting of a polypeptide, a protein, a polysaccharide, an oligosaccharide, a polyphenol and a lipid.

2. The resin composition of claim 1, which further comprises an inorganic solid.

3. The resin composition of claim 2, wherein the inorganic solid is at least one member selected from the group consisting of clays, organically modified clays, minerals and water insoluble salts.

4. The resin composition of claim 2, wherein a weight ratio of the inorganic solid to polyamide epichlorohydrin is from 1:99 to 30:70.

5. The resin composition of claim 1, wherein a weight ratio of polyamide epichlorohydrin to the macromolecule is from 1:99 to 20:80.

6. The resin composition of claim 5, wherein the macromolecule is the polyphenol.

7. A controlled release composition, comprising a plurality of particles comprising:
a core comprising at least one hydrophobic active ingredient and the resin composition of claim 1; and
a shell at least partially surrounding the core and effective to inhibit diffusion of the at least one hydrophobic active ingredient into an environment surrounding the controlled release composition.

8. A controlled release composition, comprising a plurality of particles comprising:
a core comprising at least one hydrophobic active ingredient and a macromolecule selected from the group consisting of a polypeptide, a protein, a polysaccharide, an oligosaccharide, a cellulosic material, a polyphenol and a lipid, wherein the macromolecule is crosslinked with a water-based crosslinking agent that is reactive with at least one of an amine functionality, a carboxyl functionality, hydroxyl functionality, and a thiol functionality; and
a shell at least partially surrounding the core and effective to inhibit diffusion of the at least one hydrophobic active ingredient into an environment surrounding the controlled release composition.

9. The controlled release composition of claim 8, which is a consumer product selected from the group consisting of a powdered food product, a fluid food product, a powdered nutritional supplement, a fluid nutritional supplement, a fluid fabric enhancer, a solid fabric enhancer, a fluid shampoo, a solid shampoo, a hair conditioner, a body wash, a solid antiperspirant, a fluid antiperspirant, a solid deodorant, a fluid deodorant, a fluid detergent, a solid detergent, a fluid hard surface cleaner, a solid hard surface cleaner, a fluid fabric refresher spray, a diaper, an air freshening product, a nutraceutical supplement, a controlled release fertilizer, a controlled release insecticide, a controlled release dye and a unit dose detergent further comprising a detergent and a water soluble outer film.

10. The controlled release composition of claim 8, wherein the particles have a diameter from 0.1 microns to less than 200 microns.

11. The controlled release composition of claim 8, further comprising at least one suspension agent effective to suspend the particles, wherein the at least one suspension agent is at least one member selected from the group consisting of a rheology modifier, a structurant and a thickener.

12. The controlled release composition of claim 11, wherein the at least one suspension agent has a high shear viscosity, at 20 sec⁻¹ shear rate and at 21° C., of from 1 to 7000 cps and a low shear viscosity, at 0.5 sec⁻¹ shear rate at 21° C., of greater than 1000 cps.

13. The controlled release composition of claim 11, wherein the at least one suspension agent is a member selected from the group consisting of polyacrylates, polymethacrylates, polycarboxylates, pectin, alginate, gum arabic, carrageenan, gellan gum, xanthan gum, guar gum, gellan gum, hydroxyl-containing fatty acids, hydroxyl-containing fatty esters, hydroxyl-containing fatty waxes, castor oil, castor oil derivatives, hydrogenated castor oil derivatives, hydrogenated castor wax, perfume oil, and mixtures thereof.

14. The controlled release composition of claim 8, which is a fluid having a high shear viscosity, at 20 sec⁻¹ and at 21° C., of from 50 to 3000 cps and a low shear viscosity, at 0.5 sec⁻¹ shear rate at 21° C., of greater than 1000 cps.

15. The controlled release composition of claim 8, which comprises at least two different types of friction-triggered controlled release particles effective to release the at least one hydrophobic active ingredient at different rates due to a difference in shell material friability or core material viscosity.

16. The controlled release composition of claim 8, wherein the at least one hydrophobic active ingredient comprises a mixture of a hydrophobic active and a material selected from the group consisting of brominated oils, epoxidized oils, highly nonpolar oils, hydrophobically modified inorganic particles, nonionic emulsifiers and oil thickening agents.

17. The controlled release composition of claim 8, wherein the shell is comprised of a material having an Environmental Biodegradability greater than 50%.

18. The controlled release composition of claim 8, wherein the shell is degradable by microbes found in wastewater streams to release the at least one hydrophobic active ingredient.

19. The controlled release composition of claim 8, wherein the at least one hydrophobic active ingredient is at least one member selected from the group consisting of a flavorant, a fragrance, a chromogen, a dye, an essential oil, a sweetener, an oil, a pigment, an active pharmaceutical ingredient, a moldicide, a herbicide, a fertilizer, a phase change material, an adhesive, a vitamin oil, a vegetable oil, a triglyceride and a hydrocarbon.

20. A method for preparing a composition, said method comprising the following steps:
mixing a macromolecule with a polyamide epichlorohydrin to provide a homogeneous suspension in water;
adjusting a pH of the homogenous suspension;
dehydrating the homogeneous suspension to provide a powder; and
heating the powder at a temperature greater than 100° C. for more than 30 minutes to provide the composition, which comprises the resin composition of claim 1.

21. A method for preparing a composition, said method comprising the following steps:
mixing a macromolecule with a polyamide epichlorohydrin to provide a homogeneous suspension in water;
adjusting a pH of the homogenous suspension;
dehydrating the homogeneous suspension to provide a powder; and
heating the powder at a temperature greater than 100° C. for more than 30 minutes to provide the composition, which comprises the resin composition of claim 8 and the method further comprises forming the shell by any one of: (a) condensation reactions, (b) free radical polymerization reactions, (c) interfacial polymerization reactions, or (d) coacervation of pre-formed polymers followed by crosslinking of the thereby obtained coacervates by using a crosslinker.
